(19) United States
(12) Patent Application Publication
(10) Pub. No.: US 2011/0003327 A1
(43) Pub. Date: Jan. 6, 2011

(54) METHODS FOR PRODUCTION OF ATRIAL PROGENITORS AND THEIR DIFFERENTIATION INTO SMOOTH MUSCLE CELLS AND CARDIOMYOCYTES

(75) Inventors: Kenneth Chien, Cambridge, MA (US); Atsushi Nakano, Los Angeles, CA (US); Haruko Nakano, Los Angeles, CA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/921,621

(22) PCT Filed: Mar. 12, 2009
(86) PCT No.: PCT/US09/36924

§ 371 Date: Sep. 9, 2010

(30) Foreign Application Priority Data
Mar. 14, 2008 (US) 61/036668

(51) Int. Cl. C12Q 1/02; C12N 5/10; C12N 5/071

(52) U.S. Cl. 435/29; 435/325; 435/377; 435/366

(57) ABSTRACT

The present invention generally relates to methods to identify and isolate atrial progenitors, and in some embodiments to the atrial progenitors are positive for both Islet 1 (Isl1) and sarcolipin (SLN). One aspect of the present invention relates to methods to differentiate progenitors into Isl1+/SLN+ atrial progenitors. Another aspect of the invention relates to methods to differentiate Isl1⁺/SLN⁺ atrial progenitors to smooth muscle and cardiomyocyte phenotypes. A further aspect of the invention relates to reprogramming postnatal and mature atrial myocytes to atrial progenitors positive for Isl1+/SLN+, and the subsequent differentiation of Isl1⁺/SLN+ atrial progenitors to smooth muscle and cardiomyocyte phenotypes. Another aspect of the invention relates to a composition comprising an isolated population of Islet1⁺, SLN⁺ atrial progenitor cells, and uses thereof.

### CROSS REFERENCE TO RELATED APPLICATIONS

This application claims priority under 35 U.S.C. 119(e) of U.S. Provisional Patent Application Ser. No. 61/036,668 filed 14 Mar. 2008, the contents of which are incorporated herein by reference in their entirety.

### GOVERNMENT SUPPORT

This invention was made with Government Support under a grant awarded by the National Institutes of Health. The Government has certain rights thereto.

### FIELD OF THE INVENTION

The present invention relates generally to methods to identify and isolate atrial progenitors, in particular atrial progenitors positive for Isl1⁺/SLN+. The present invention also relates to methods to differentiate Isl1⁺/SLN+ atrial progenitors to smooth muscle and cardiomyocyte phenotypes. The present invention also relates to reprogramming postnatal and mature atrial myocytes to atrial progenitors positive for Isl1+/SLN+, and subsequent differentiation of Isl1⁺/SLN+ atrial progenitors to isolation smooth muscle and cardiomyocyte phenotypes.

### BACKGROUND

Cardiovascular disease involves diseases or disorders associated with the cardiovascular system. Such disease and disorders include those of the pericardium, heart valves, myocardium, blood vessels, and veins.

Over the last two decades, the morbidity and mortality of heart failure has markedly increased (Tavazzi, 1998). Therefore, finding an effective therapeutic method is one of the greatest challenges in public health for this century. Although there are several alternative ways for treatment of heart failure, such as coronary artery bypass grafting and whole-heart transplantation, myocardial fibrosis and organ shortage, along with strict eligibility criteria, mandate the search for new approaches to treat the disease. Cell transplantation has emerged to be able to increase the number of contractile myocytes in damaged hearts. However, cardiomyocytes, which are also known as cardiac muscle cells, are terminally differentiated cells and are unable to divide and their use in cell transplantation is limited by the inability to obtains sufficient quantities of cardiomyocytes for the repair of large areas of infarct myocardium. Thus, alternative sources of cells for cell transplantation need to be used, such as stem cells. However, the use of using non-committed stem cells also possess the risk of their differentiation into non-cardiac cells and risk of teratomas post transplantation.

Thus, there is a need in the art to develop alternatives to the presently used cells and transplantation techniques for the treatment of cardiovascular disease.

The generation of diverse endothelial, smooth muscle, and cardiac cell lineages in discrete heart chambers and vessels is a critical step in cardiogenesis. Multipotent Isl1⁺ and other heart progenitors play a pivotal role in lineage diversification, giving rise to all three of these major cell types in both the vessels and cardiac chambers¹⁻⁶. A critical question is pinpointing when this cardiac-vascular lineage decision is made, how this bi-potency serves to coordinate cardiac chamber and vessel growth, and determining to what extent these steps are irreversible.

### SUMMARY

The present invention relates to methods for the production of atrial progenitors cells. In some embodiments, atrial progenitor cells are positive for Islet 1 (Isl1) and also positive for atrial-specific sarcolipin (SLN), and are referred to herein as “Isl1⁺/SLN⁺ atrial progenitors”. In some embodiments, the Isl1⁺/SLN⁺ atrial progenitors can be derived from the reprogramming of differentiated cardiomyocytes (such as for example, atrial myocytes) to an earlier developmental stage to become Isl1⁺/SLN⁺ atrial progenitors. In some embodiments, the cardiomyocyte-derived Isl1⁺/SLN⁺ atrial progenitors are derived from the reprogramming of postnatal myocardial atrial myocytes. In alternative embodiments Isl1⁺/SLN⁺ atrial progenitors can be derived by the differentiation of immature cardiac progenitors such as cardiac progenitors that express Isl1⁺ but are negative for the expression of SLN.

By utilizing lineage tracing with an atrial-specific sarcolipin (SLN) Cre line of knock-in mice, the inventors have discovered a population of atrial progenitors which are Isl1⁺/SLN⁺ atrial progenitors which can differentiate into cardiac muscle and/or smooth muscle in the boundary of the myocardial and smooth muscle layer in the inflow tract of the heart. The inventors have discovered that a single Isl1⁺/SLN⁺ atrial progenitor cell can be clonally expanded and differentiated into both cardiac and smooth muscle cells. While atrial progenitors progressively lose smooth muscle cell competence during cardiogenesis, postnatal atrial myocytes, for example atrial myocytes that are Isl1⁻/cTnT⁺/MLC2v⁻/SLN⁺ can, upon re-exposure to the cardiac mesenchymal feeder layer, be reprogrammed to re-express Isl1, re-enter the cell cycle, and then trans-differentiate into vascular smooth muscle cells. The inventors demonstrate with studies with MLC2v cre knock-in mice that this reversible bi-potency is specific for atrial Isl1⁺/SLN⁺ progenitors.

Accordingly, the inventors have demonstrated that Isl1⁺/SLN⁺ atrial progenitors, a subpopulation of cardiac progenitors during cardiogenesis, can display reversible bipotency even after birth, and that bi-potency of atrial progenitors coordinates junctional morphogenesis between the cardiac chambers and great veins. The inventors have discovered the role of defects in the control of bipotency in the onset of atrial and inflow tract diseases, and the potential utility of the reprogramming of post-natal atrial Isl1 progenitors as a foundation for regenerative cardiovascular therapies for the newborn heart⁷.

One aspect of the present invention relates to the identification of atrial progenitors which are positive for both Isl1 and SNL.

Another aspect of the present invention relates to the induction of cells to become Isl1⁺/SLN⁺ atrial progenitors, for example the reprogramming of mature atrial myocytes to become Isl1⁺/SLN⁺ atrial progenitors.

Another aspect of the present invention relates to the differentiation of Isl1⁺/SLN⁺ atrial progenitors to different muscle phenotypes, for example for their differentiation to mature atrial myocyte cells such as mature cardiomyocytes which are Isl1⁻/cTnT⁺/MLC2v⁻/SLN⁺, or to mature smooth muscle cells, for example smooth muscle cells which are Isl1⁻/cTnT⁻/SLN⁻/smMHC⁺.

### BRIEF DESCRIPTION OF FIGURES

FIG. 1A-1C shows atrial lineage tracing. FIG. 1A shows a schematic of SLN genomic locus, targeting vector design and recombinant alleles. Exon 2 which including the start codon was replaced by Cre recombinase and neomycin resistant cassette by homologous recombination. FRT sites are indicated by filled triangles. DTA, diphtheria toxin A cassette. FIG. 1B shows a genomic Southern blot analysis of targeted ES cells and heterozygous mouse after removal of neo cassette using 5′ and 3′ probe shown in FIG. 1A. FIG. 1C shows a genomic PCR for mouse genotyping. Primer designs are shown in FIG. 1A.

FIG. 2A-2B shows the bipotency potential of atrial progenitors. FIG. 2A shows clonal amplification and differentiation of single atrial progenitors. Atrial progenitors are isolated from the atria dissected from SLN^{cre/+}×R26R embryos at E9.5, and cultured on cardiac mesenchymal feeder at clonal density. They form colonies and maintain Isl1 expression on a cardiac mesenchymal feeder (left panels). Differentiated colonies were stained for Xgal, cTnT and/or smMHC (right panels). Most of the cells in Xgal-positive colonies were positive for cTnT, but some in the periphery were negative (black arrows). These peripheral cells are co-stained with Xgal and smMHC (white arrows). Right bottom panels show immunofluorescent staining for cTnT and smMHC. Boxed area of far left panel is enlarged in the far right panel to show the distinction of cTnT-positive and smMHC-positive cells (white arrow). Scale bar=50 μm. FIG. 2B shows the expression profile of atrial progenitor colonies. After 3-4 (early stage) and 7-12 days (late stage) on feeders, colonies were picked up and examined for marker expression, showing that Isl1 is positive at early stage and that smMHC become positive in half of the colonies. DsRed-labeled atrial cells at late stage were sorted and replated on glass slides by cytospin. Immunostaining showed that 96.6% and 3.1% of the sorted cells were positive for cTnT and smMHC, respectively.

FIG. 3A-3E shows reprogramming of postnatal atrial myocytes. FIG. 3A shows quantitative analysis of Histone H3 trimethylation levels by ChIP-qPCR assay. H3K27me3 trimethylation level is lower after Isl1 re-activation, suggesting that Isl1 re-activation is due to epigenetic activation of Isl1 promoter activity. FIG. 3B shows Isl1-positive atrial cells redifferentiate into smooth muscle cells and ventricular myocytes. Atrial myocytes are isolated from Isl1mCm/+×R26R breeding and treated with 4OH-TAM for 48 hours starting from day 2 on the culture dish. The labeled cells were further differentiated and analyzed for marker expressions. By calculation, 97.6% of the Xgal-positive cells are supposed to be derived from atrial myocytes. FIG. 3C shows [Ca2+]i transient assay of the atrial myocyte-derived smooth muscle cells. DsRed-labeled atrial myocytes were stimulated with Angiotensin-II. 3 of 30 cells responded in a pattern similar to cultured aortic smooth muscle cells. Right upper panel shows [Ca2+]i oscillation typically seen in atrial myocytes that did not acquire smooth muscle phenotype. Right lower panel shows [Ca2+]i transient of aortic smooth muscle cells employed as a control. FIG. 3D shows a model for smooth muscle cell contribution of atrial progenitors during migration from the splanchnic mesoderm. FIG. 3E shows a model for reprogramming of postnatal atrial myocytes to Isl1+ progenitor-like state.

FIG. 4 shows quantitative analysis of Isl1 mRNA level in genetic ablation models. In Ryr2 conditional knockout experiment, 4-week-old αMHC; Ryr2flox/flox mice (Ryr2 CKO) and their control littermates (Ryr2flox/flox mice; cont) were intraperitoneally injected with TAM to induce acute cardiac damage 3 days prior to the RNA extraction from atrial appendages. In MLP experiment, MLP mutant mice (MLP) and their control wild-type littermates (cont) were analyzed. MLP mice are functionally normal by 4-8 wks old and gradually develop heart failure and 4-chamber dilation thereafter. Error bars indicate S.D.

FIG. 5 shows quantitative analysis of Isl1 and Nkx2.5 mRNA levels by qPCR using RNA from atrial myocyte, ventricular myocyte and the cardiac mesenchymal fibroblast fraction isolated from neonatal heart. Whereas Nkx2.5 is equally expressed in atrial and ventricular myocytes, the Isl1 level in atrial myocytes is 18-fold higher than ventricular myocytes and 4-fold higher than cardiac mesenchymal fibroblasts. If we consider that the contamination of fibroblast in myocyte fraction is 10% and that the contamination of myocyte in fibroblast fraction is 1%, Isl1 mRNA level in primary atrial myocyte (AM) fraction and cardiac fibroblast (CF) fraction is:

AM fraction; 0.9x+0.1y=1.90 - - - (a)
CF fraction; 0.01x+0.99y=0.48 - - - (b)

where x is Isl1 level in genuine atrial cardiomyocyte and y is Isl1 level in messenchyme (residential Isle1 progenitor ganglion cells, etc). Simultaneous equation (a) and (b) is solved in this way:

(a)×9.9; 8.91x+0.99y=18.81 - - - (c)

(c)−(b); 8.90x=18.33
Therefore x=2.06

(a); 0.9×2.06+0.1y=1.90
Therefore y=0.46

Therefore, the percentage of myocyte-derived Isl1 in atrial myocyte fraction is;

0.9x/(0.9X+0.1y)×100(%)=97.6(%)

These calculation indicates that most (97.6%) of the Isl1 mRNA in atrial myocyte fraction is derived from genuine atrial cardiomyocyte.

### DETAILED DESCRIPTION

As disclosed herein, the inventors have discovered a population of atrial progenitors which are positive for the markers Isl1⁺ and SNL have a biopotency potential to differentiate into cardiomyocytes and smooth muscle cell phenotypes. Further, the inventors have also discovered that postnatal and mature atrial myocytes can be reprogrammed to an earlier developmental stage and can become atrial progenitors which are Isl1⁺/SLN⁺. The inventors have discovered that such atrial myocyte derived Isl1⁺/SLN⁺ atrial progenitors have the capacity to differentiate into cardiomyocytes and smooth muscle cell phenotypes, and such retro-differentiated atrial myocytes have the capacity to reenter cell cycle into atrial lineages. Therefore, the inventors have discovered that mature and postnatal atrial myocytes can be induced to become Isl1⁺/SLN⁺ atrial progenitors and subsequently differentiated into cardiaomyocytes and/or distinct muscle phenotypes, which can be transplanted into a subject for the treatment and/or prevention of cardiac diseases, or for the treatment of existing cardiac muscle which is damaged by disease or injury.

Herein, utilizing lineage tracing with an atrial-specific sarcolipin (SLN)-Cre line of knock-in mice, the inventors demonstrate the discovery of Isl1+/SLN+ atrial progenitors that contribute to cardiac as well as smooth muscle in the boundary of the myocardial and smooth muscle layer in the inflow tract of the heart. The inventors demonstrate that single Isl1+/SLN+ atrial progenitors can be clonally expanded and differentiated into both cardiac and smooth muscle cells. The inventors also demonstrate that the inhibition of the differentiation of atrial progenitors resulted in the defects in anchoring atrium and inflow tract. While atrial progenitors progressively lose smooth muscle cell competence during cardiogenesis, the inventors demonstrate that post-natal atrial myocytes, upon re-exposure to the cardiac mesenchymal feeder layer, can be reprogrammed to re-express Isl1, re-enter the cell cycle, and then redifferentiate into vascular smooth muscle cells as well as ventricular myocytes that can be engrafted into the ventricular wall. The inventors have discovered that defects in the control of biopotency can lead to the onset of atrial and inflow tract diseases, and that reprogrammed Isl1 progenitors can be used in regenerative cardiovascular therapies for the newborn heart⁷.

Accordingly, one aspect of the present invention relates to a methods for identifying and selecting for atrial progenitors in a population of cells, for example cardiovascular stem cells or a population of atrial myocytes, involving contacting the population of cells with agent which are reactive to Islet 1 (Isl1) and atrial-specific sarcolipin (SLN) and isolating the positive cell from the non-reactive cells. In some embodiments, the agents are reactive to nucleic acids and in other embodiments the agents are reactive to the proteins expressed by the Isl1⁺ and SLN genes. Another embodiment comprises isolating and identifying the atrial progenitors expressing Isl1⁺ and SLN⁺ using conventional methods of using a marker gene operatively linked to a promoter of Isl1⁺ and/or SLN⁺.

Another aspect of the present invention relates to the induction of mature or postnatal atrial myocytes, such as a mature atrial myocyte cell that is Isl1⁺/cTNT⁺/MLC2v⁻/SLN⁺ to a Isl1⁺/SLN⁺ atrial progenitor phenotype. In one embodiment, the present invention relates to methods of inducing a mature atrial myocyte, such as a Isl1⁺/cTNT⁺/MLC2v⁻/SLN⁺ atrial myocyte to become an earlier developmental stage and becoming an Isl1⁺/SLN⁺ atrial progenitor. The process of a cell reverting to an earlier developmental stage is commonly known in the art and is referred to herein as “reprogramming.”

In one embodiment, the present invention provides a method of reprogramming a mature atrial myocyte to Isl1⁺/SLN⁺ atrial progenitor, the method comprising culturing the atrial myocytes on a messenchymal feeder layer, for example a cardiac messenchymal fibroblast feeder layer. In some embodiments, the mature atrial myocytes that are induced along a reprogramming pathway to become Isl1⁺/SLN⁺ atrial progenitors are postnatal Isl1⁺/cTNT⁺/MLC2v⁻/SLN⁺ atrial myocytes, and in some embodiments, they are adult Isl1⁺/cTNT⁺/MLC2v⁻/SLN⁺ atrial myocytes. In some embodiments, the atrial myocytes are from a mammalian subject, for example a human subject.

Another aspect of the present invention relates to the differentiation of Isl1⁺/SLN⁺ atrial progenitor cells into cardiomyocytes and smooth muscle myocytes, for example postnatal or mature atrial myocytes.

Another aspect of the present invention relates to methods for the use of Isl1⁺/SLN⁺ atrial progenitors, for example atrial myocyte derived Isl1⁺/SLN⁺ atrial progenitors. In some embodiments, the Isl1⁺/SLN⁺ atrial progenitors can be used for the production of a pharmaceutical composition, for example, for the transplantation into a subject in need of cardiac regenerative therapy, for example subjects with congenital heart diseases as well as subjects with acquired congenital defects or diseases, such as, for example cardiac muscle which is damaged by disease or injury. In some embodiments, subject amenable to treatment with the pharmaceutical composition as disclosed herein include, for example congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “atrial progenitor” as used herein, refers to a progenitor cell which is capable of proliferation and giving rise to more progenitor cells having the ability to generate a large number of mother cells that can in turn give rise to differentiated, or differentiable daughter cells which can eventually terminally differentiate primarily into cardiomyocytes, for example, but not limited smooth muscle cells and/or myocardial cells such as atrial myocytes.

The term “cardiomyocyte” as used herein broadly refers to a muscle cell of the heart. The term cardiomyocyte includes smooth muscle cells of the heart, as well as cardiac muscle cells, which include also include striated muscle cells, as well as spontaneous beating muscle cells of the heart.

As used herein, the term “Isl1” refers to the nucleic acid encoding Islet 1 gene and homologues thereof, including conservative substitutions, additions, deletions therein not adversely affecting the structure of function. Isl1 is referred in the art as Islet 1, ISL LIM homeobox 1 or Isl-1. Human Isl1 is encoded by nucleic acid corresponding to GenBank Accession No: BC031213 (amino acid and nucleotide sequences disclosed as SEQ ID NOS 1 and 2, respectively) or NM_{—}002202 (amino acid and nucleotide sequences disclosed as SEQ ID NOS 1 and 3, respectively) and the human Isl1 corresponds to protein sequence corresponding to RefSeq ID No: AAH31213 (SEQ ID NO:1)

As used herein, the term “SLN” refers to the nucleic acid encoding the atrial-specific sarcolipin gene and homologues thereof, including conservative substitutions, additions, deletions therein not adversely affecting the structure or biological function of SLN. Human SLN is encoded by nucleic acid corresponding to GenBank Accession No: U96094 (amino acid and nucleotide sequences disclosed as SEQ ID NOS 4 and 5, respectively) or NM_{—}003063 (amino acid and nucleotide sequences disclosed as SEQ ID NOS 4 and 6, respectively) or Gene ID: 6588 (SEQ ID NO: 6), which the human SLN cDNA encodes a protein of 31 amino acids and corresponds to protein sequence of RefSeq ID No: AAB86981 (SEQ ID NO: 4).

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “reprogramming” as that them is defined herein.

The term “progenitor cells” is used synonymously with “stem cell.” Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate. It is possible that cells that begin as progenitor cells might proceed toward a differentiated phenotype, but then “reverse” and re-express the progenitor cell phenotype.

The term “reprogramming” as used herein refers to the transition of a differentiated cell to become a progenitor cell. Stated another way, the term reprogramming refers to the transition of a differentiated cell to an earlier developmental phenotype or developmental stage. A “reprogrammed cell” is a cell that has reversed or retraced all, or part of its developmental differentiation pathway to become a progenitor cell. Thus, a differentiated cell (which can only produce daughter cells of a predetermined phenotype or cell linage) or a terminally differentiated cell (which can not divide) can be reprogrammed to an earlier developmental stage and become a progenitor cell, which can both self renew and give rise to differentiated or undifferentiated daughter cells. The daughter cells themselves can be induced to proliferate and produce progeny that subsequently differentiate into one or more mature cell types, while also retaining one or more cells with parental developmental potential. The term reprogramming is also commonly referred to as retrodifferentiation or dedifferentiation in the art. A “reprogrammed cell” is also sometimes referred to in the art as an “induced pluripotent stem” (IPS) cell.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an atrial precursor), and then to an end-stage differentiated cell, such as atrial cardiomyocytes or smooth muscle cells which plays a characteristic role in a certain tissue type, and may or may not retain the capacity to proliferate further.

As indicated above, there are different levels or classes of cells falling under the general definition of a “stem cell.” These are “totipotent,” “pluripotent” and “multipotent” stem cells. The term “totipotent” refers to a stem cell that can give rise to any tissue or cell type in the body. “Pluripotent” stem cells can give rise to any type of cell in the body except germ line cells. Stem cells that can give rise to a smaller or limited number of different cell types are generally termed “multipotent.” Thus, totipotent cells differentiate into pluripotent cells that can give rise to most, but not all, of the tissues necessary for fetal development. Pluripotent cells undergo further differentiation into multipotent cells that are committed to give rise to cells that have a particular function. For example, multipotent hematopoietic stem cells give rise to the red blood cells, white blood cells and platelets in the blood.

The term “differentiation” in the present context means the formation of cells expressing markers known to be associated with cells that are more specialized and closer to becoming terminally differentiated cells incapable of further differentiation. The pathway along which cells progress from a less committed cell, to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated. Differentiation is a developmental process whereby cells assume a more specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, we note that in the context of this specification, the terms “differentiation” or “differentiated” refer to cells that are more specialized in their fate or function than at one time in their development. For example in the context of this application, a differentiated cell includes a cell differentiated from an Isl1⁺/SLN⁺ atrial progenitor where such Isl1⁺/SLN⁺ atrial progenitor is derived from the reprogramming of a mature atrial myocytes. Thus, while such a differentiated cell is more specialized than the time in which it had the phenotype of an Isl1⁺/SLN⁺ atrial progenitor, it can also be less specialized as compared to when it existed as a mature atrial myocyte (prior to its reprogramming to an Isl1⁺/SLN⁺ atrial progenitor). Accordingly, a differentiated cell as used herein can be more specialized than a Isl1⁺/SLN⁺ atrial progenitor, but more or less specialized than a mature cardiomyocte cell from which the Isl1⁺/SLN⁺ atrial progenitor was derived.

The term “enriching” is used synonymously with “isolating” cells, and means that the yield (fraction) of cells of one type is increased over the fraction of cells of that type in the starting culture or preparation.

The development of a cell from an uncommitted cell (for example, a stem cell), to a cell with an increasing degree of commitment to a particular differentiated cell type, and finally to a terminally differentiated cell is known as progressive differentiation or progressive commitment. A cell that is “differentiated” relative to a progenitor cell has one or more phenotypic differences relative to that progenitor cell. Phenotypic differences include, but are not limited to morphologic differences and differences in gene expression and biological activity, including not only the presence or absence of an expressed marker, but also differences in the amount of a marker and differences in the co-expression patterns of a set of markers.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

A “marker” as used herein describes the characteristics and/or phenotype of a cell. Markers can be used for selection of cells comprising characteristics of interest. Markers vary with specific cells. Markers are characteristics, whether morphological, functional or biochemical (enzymatic) characteristics particular to a cell type, or molecules expressed by the cell type. Preferably, such markers are proteins, and more preferably, possess an epitope for antibodies or other binding molecules available in the art. A marker may consist of any molecule found in, or on the surface of a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method commonly available to one of skill in the art.

A “reporter gene” as used herein encompasses any gene that is genetically introduced into a cell that adds to the phenotype of the stem cell. Reporter genes as disclosed in this invention are intended to encompass fluorescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny.

The term “lineages” as used herein refers to a term to describe cells with a common ancestry, for example cells that are derived from the same cardiovascular stem cell or other stem cell.

As used herein, the term “clonal cell line” refers to a cell lineage that can be maintained in culture and has the potential to propagate indefinitely. A clonal cell line can be a stem cell line or be derived from a stem cell, and where the clonal cell line is used in the context of a clonal cell line comprising stem cells, the term refers to stem cells which have been cultured under in vitro conditions that allow proliferation without differentiation for months to years. Such clonal stem cell lines can have the potential to differentiate along several lineages of the cells from the original stem cell.

The term “phenotype” refers to one or a number of total biological characteristics that define the cell or organism under a particular set of environmental conditions and factors, regardless of the actual genotype.

The terms “mesenchymal cell” or “mesenchyme” are used interchangeably herein and refer in some instances to the fusiform or stellate cells found between the ectoderm and endoderm of young embryos; most mesenchymal cells are derived from established mesodermal layers, but in the cephalic region they also develop from neural crest or neural tube ectoderm. Mesenchymal cells have a pluripotential capacity, particularly embryonic mesenchymal cells in the embryonic body, developing at different locations into any of the types of connective or supporting tissues, to smooth muscle, to vascular endothelium, and to blood cells.

The term “tissue” refers to a group or layer of similarly specialized cells which together perform certain special functions. The term “tissue-specific” refers to a source or defining characteristic of cells from a specific tissue.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “enriching” or “enriched” are used interchangeably herein and mean that the yield (fraction) of cells of one type is increased by at least 10% over the fraction of cells of that type in the starting culture or preparation.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, refer to a population of cells that contain fewer than about 20%, more preferably fewer than about 15%, 10%, 8%, 7%, most preferably fewer than about 5%, 4%, 3%, 2%, 1%, or less than 1%, of cells that are not stem cells or stem cell progeny.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” or “recombinant gene” refers to a nucleic acid comprising an open reading frame encoding a polypeptide, including both exon and (optionally) intron sequences.

The term “recombinant,” as used herein, means that a protein is derived from a prokaryotic or eukaryotic expression system.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g EPV and EBV vectors.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “regulatory sequence” and “promoter” are used interchangeably herein, refers to a generic term used throughout the specification to refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

The terms “subject” and “individual” are used interchangeably herein, and refer to an animal, for example a human, to whom treatment, including prophylactic treatment, with methods and compositions described herein, is or are provided. For treatment of those infections, conditions or disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates.

The term “regeneration” means regrowth of a cell population, organ or tissue after disease or trauma.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “disease” or “disorder” is used interchangeably herein, and refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition or affection.

The term “pathology” as used herein, refers to symptoms, for example, structural and functional changes in a cell, tissue or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

As used herein, the terms “treat” or “treatment” or “treating” refers to both therapeutic treatment and prophylactic or preventative measures, wherein the object is to prevent or slow the development of the disease, such as slow down the development of a cardiac disorder, or reducing at least one adverse effect or symptom of a cardiovascular condition, disease or disorder, i.e., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “effective amount” as used herein refers to the amount of therapeutic agent of pharmaceutical composition to reduce at least one or more symptom(s) of the disease or disorder, and relates to a sufficient amount of pharmacological composition to provide the desired effect. The phrase “therapeutically effective amount” as used herein, e.g., of population of atrial progenitors or atrial myocytes as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylatically significant reduction in a symptom or clinical marker associated with a cardiac dysfunction or disorder when administered to a typical subject who has a cardiovascular condition, disease or disorder.

A therapeutically or prophylatically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

With reference to the treatment of a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick J P, Am J Physiol Heart circ Physiol 285; H1797; 2003) and animal models acute myocardial infarction. (Yang Z, Am J Physiol Heart Circ. Physiol 282:H949:2002; Guo Y, J Mol Cell Cardiol 33; 825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals. By “earlier” is meant that a decrease, for example in the size of the tumor occurs at least 5% earlier, but preferably more, e.g., one day earlier, two days earlier, 3 days earlier, or more.

As used herein, the term “treating” when used in reference to a cancer treatment is used to refer to the reduction of a symptom and/or a biochemical marker of cancer, for example a reduction in at least one biochemical marker of cancer by at least about 10% would be considered an effective treatment. Examples of such biochemical markers of cardiovascular disease include a reduction of, for example, creatine phosphokinase (CPK), aspartate aminotransferase (AST), lactate dehydrogenase (LDH) in the blood, and/or a decrease in a symptom of cardiovascular disease and/or an improvement in blood flow and cardiac function as determined by someone of ordinary skill in the art as measured by electrocardiogram (ECG or EKG), or echocardiogram (heart ultrasound), Doppler ultrasound and nuclear medicine imaging. A reduction in a symptom of a cardiovascular disease by at least about 10% would also be considered effective treatment by the methods as disclosed herein. As alternative examples, a reduction in a symptom of cardiovascular disease, for example a reduction of at least one of the following; dyspnea, chest pain, palpitations, dizziness, syncope, edema, cyanosis etc. by at least about 10% or a cessation of such systems, or a reduction in the size one such symptom of a cardiovascular disease by at least about 10% would also be considered as affective treatments by the methods as disclosed herein. In some embodiments, it is preferred, but not required that the therapeutic agent actually eliminate the cardiovascular disease or disorder, rather just reduce a symptom to a manageable extent.

Subjects amenable to treatment by the methods as disclosed herein can be identified by any method to diagnose myocardial infarction (commonly referred to as a heart attack) commonly known by persons of ordinary skill in the art are amenable to treatment using the methods as disclosed herein, and such diagnostic methods include, for example but are not limited to; (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

As used herein, the terms “administering,” “introducing” and “transplanting” are used interchangeably and refer to the placement of the cardiac myocytes as described herein into a subject by a method or route which results in at least partial localization of the cardiovascular stem cells at a desired site. The cardiovascular stem cells can be administered by any appropriate route which results in effective treatment in the subject, i.e. administration results in delivery to a desired location in the subject where at least a portion of the cells or components of the cells remain viable. The period of viability of the cells after administration to a subject can be as short as a few hours, e.g. twenty-four hours, to a few days, to as long as several years.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of atrial progenitors or atrial myocytes and/or their progeny and/or compound and/or other material other than directly into the cardiac tissue, such that it enters the animal's system and, thus, is subject to metabolism and other like processes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

The term “drug” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “agent” refers to any entity which is normally not present or not present at the levels being administered to a cell, tissue or subject. Agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An gent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean ±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Methods for Identification and Isolating Isl1+/SLN+ Atrial Progenitor Cells

In the present invention, an atrial progenitor cell has been discovered, isolated and characterized. One aspect of the invention provides methods for the isolation of a subset of atrial progenitor cells that are capable of differentiating into multiple different lineages, such as for example, smooth muscle cells and cardiomyocytes such as atrial myocytes. In particular, the invention provides methods for isolating atrial progenitor cells capable of contributing to the majority of muscle cells and a cardiomyocytes in the heart. These atrial progenitor cells are positive for Islet1 (Isl1) and SLN markers. In one aspect, the invention relates to methods of isolation of these atrial progenitor cells, and another aspect relates to their differentiation into smooth muscle and cardiomyocytes in the heart. Encompassed in the invention are methods for the identification and isolation of such atrial progenitor cells by the agents that are reactive to Islet1 (Isl1) and SLN, including agents reactive to the nucleic acids encoding Islet1 (Isl1) and SLN.

In another embodiment, agents reactive to the expression products of the Islet1-(Isl1) and SLN-encoding nucleic acids, for example agents reactive to Isl1, SLN proteins or polypeptides, or fragments thereof. Another embodiment encompasses methods for the identification and isolation of atrial progenitor cells comprising Isl1 and SLN markers using a marker gene operatively linked to promoters of Isl1 and/or SLN, or homologues or variants thereof.

In some embodiments, at least some of the cardiovascular stem cells also comprise or more selected to comprise additional markers, for example the heart-associated transcription factors. In one embodiment, the invention relates to a method of isolating populations of atrial progenitor cells characterized by the markers Isl-1 and SLN1 by means of positive selection. The methods described permit enrichment of a purified population or substantially pure population expressing Isl-1 and SLN to be obtained.

### Differentiation of Isl1⁺/SLN⁺ Atrial Progenitors

In some embodiments, the Isl1⁺/SLN⁺ atrial progenitor cells differentiate along different lineages; therefore these atrial progenitor cells have multi-linage differentiation potential. In one embodiment, the Isl1⁺/SLN⁺ atrial progenitor cells differentiate into smooth muscle cells. In one embodiment, the smooth muscle cells resulting from such differentiation are positive for markers smMHC (smMHC⁺), and negative for Isl1 (Isl1⁻), cTnT (cTnT⁻) and SLN (SLN⁻).

In other embodiments, the Isl1⁺/SLN⁺ atrial progenitor cells differentiate into cardiomyocytes. In some embodiments, the cardiomyocytes are atrial cardiomyocytes. In some embodiments, the cardiomyocytes resulting from such differentiation of Isl1⁺/SLN⁺ atrial progenitor cells are positive for markers cTNT (cTnT⁺), SLN (SLN⁺), and negative for isl1 (Isl1⁺) and MLC2v (MLC2v⁻).

In a further embodiment, the Isl1⁺/SLN⁺ atrial progenitor cells as described herein differentiate into smooth muscle cells of the heart and/or cardiomyocytes. Methods for such directed differentiation protocols are well known in the art, and include as a non-limiting example, directed differentiation of Isl1⁺/SLN⁺ atrial progenitor cells into cardiomyocytes can be performed by culturing the Isl1⁺/SLN⁺ atrial progenitor cells in the presence of cardiac messenchymal feeder layer cells. In alternative embodiments, the Isl1⁺/SLN⁺ atrial progenitor cells can be directed to differentiate into cardiomyocytes by culturing the cells on fibronectin coated plates in the presence of DMEM/M199 (4:1 ratio) medium containing 10% horse serum and 5% fetal bovine serum (FBS).

In alternative embodiments, the Isl1⁺/SLN⁺ atrial progenitor cells can be directed to differentiate into smooth muscle cells by culturing the progenitors in the presence of a cardiac messenchymal feeder layer. Alternatively, a non-limiting example, the Isl1⁺/SLN⁺ atrial progenitor cells stem cells can be directed to differentiate into smooth muscle cells by culturing on fibronectin in the presence of DMEM/F12 media containing B27 media and 2% FBS and 10 ng/ml EGF.

The Isl1⁺/SLN⁺ atrial progenitor cells can be differentiated into either smooth muscle cells or cardiomyocytes by culturing them in the presence of a cardiac messenchymal feeder layer. For example, the Isl1⁺/SLN⁺ atrial progenitor cells can be cultured on a separate surface to the cardiac messenchymal cell feeder layer, i.e. the Isl1⁺/SLN⁺ atrial progenitors can be on a surface above or below the cardiac messenchymal feeder layer, or alternatively the Isl1⁺/SLN⁺ atrial progenitors can be cultured in the presence of culture media obtained from the cardiac messenchymal feeder layer. Alternatively, the Isl1⁺/SLN⁺ atrial progenitors can be cultured as a monolayer within the feeder cells layer.

One important embodiment of the invention encompasses the differentiation of the Isl1⁺/SLN⁺ atrial progenitors as disclosed herein into cardiomyocytes linage cells. The cardiomyocyte lineage cells may be cardiomyocyte atrial cells, or differentiated cardiomyocytes. Differentiated cardiomyocytes include one or more of primary cardiomyocytes, nodal (pacemaker) cardiomyocytes; conduction cardiomyocytes; and working (contractile) cardiomyocytes, which may be of atrial or ventricular type. As disclosed herein in the Examples, the Isl1⁺/SLN⁺ atrial progenitors as disclosed herein can differentiate into 2 different lineages; smooth muscle cell and cardiomyocytes. As demonstrated in the Examples, Isl1⁺/SLN⁺ atrial progenitors as disclosed herein can differentiate into atrial myocytes co-expressing cTNT (cTnT⁺), SLN (SLN⁺), and negative for isl1 (Isl1⁺) and MLC2v (MLC2v⁻). In some embodiments, Isl1⁺/SLN⁺ atrial progenitors as disclosed herein can be induced to differentiate along cardiomyocyte lineages by growing on fibronectin in the presence of DMEM/MM199 (1:4 ratio) in 10% horse serum and 5% FBS, as disclosed in the examples addition of cardiotrophic factors such as those disclosed in U.S. Patent application 2003/0022367 which is incorporated herein by reference, activin A, activin B, IGF, BMPs, FGF, PDGF, LIF, EGF, TGFα, cripto gene and other growth factors known by persons of ordinary skill in the art that can differentiate cells along a cardiac muscle lineages.

Further, as demonstrated in the Examples, the Isl1⁺/SLN⁺ atrial progenitors as disclosed herein can differentiate into smooth muscle cells, which can be identified by expressing markers smooth muscle actin (SMA or SM-actin) or smooth muscle myosin heavy chain (SM-MHC) and response to vasoactive hormone Angotensin II to result in a progressive cytosolic [Ca2⁺]ᵢ increase. As demonstrated in Examples, Isl1⁺/SLN⁺ atrial progenitors as disclosed herein can also differentiate into cardiac smooth muscle cells expressing smMHC (smMHC⁺), and negative for Isl1 (Isl1⁻), cTnT (cTnT⁻) and SLN (SLN⁻). Such cardiac smooth muscle cells can differentiate into subsets of cardiomyocytes such as pacemaker, sino-atrial (SA) node and atrial-ventricular (AV) node as identified by acetylcholinesterase (Ach-esterase) as demonstrated in the Examples. The identification of Isl1⁺/SLN⁺ atrial progenitors as disclosed herein differentiated into cardiomyocytes can be identified by expressing troponin (TnT), TnT1, α-actinin, atrial natruic factor (ANT), acetylcholinesterase.

Without wishing to be bound by theory, using morphological and electrophysiological criteria, four main phenotypes of cardiomyocytes that arise during development of the mammalian heart can be distinguished: primary cardiomyocytes; nodal cardiomyocytes; conducting cardiomyocytes and working cardiomyocytes. Morphologically and functionally, the chamber myocardium of the developing atria and ventricles are distinguished from the primary myocardium of the linear heart tube. The chamber myocardium becomes trabeculated, whereas the primary myocardium is smooth and covered with cardiac cushions. The clearest markers that in mammals identify the developing chamber myocardium are the atrial natriuretic factor (Anf) and Cx40 genes, which are not expressed in the myocardium of the primary heart tube. During further development, the smooth-walled dorsal atrial wall (comprising the pulmonary and caval myocardium) as well as the atrial septa, are incorporated into the atria. These components do not express Anf, but do express Cx40. A gene that is clearly upregulated in the cardiac chambers is sarco-endoplasmic reticulum Ca2+ATPase (Serca2a), but because it is also expressed in the primary myocardium it is less suited as a marker for the developing chambers. The functional significance of the chamber program of gene expression is that it allows fast, synchronous contractions. All cardiomyocytes have sarcomeres and a sarcoplasmic reticulum (SR), are coupled by gap junctions, and display automaticity. Cells of the primary heart tube are characterized by high automaticity, low conduction velocity, low contractility, and low SR activity. This phenotype largely persists in nodal cells. In contrast, atrial and ventricular working myocardial cells display virtually no automaticity, are well coupled intercellularly, have well developed sarcomeres, and have a high SR activity. Conducting cells from the atrioventricular bundle, bundle branches and peripheral ventricular conduction system have poorly developed sarcomeres, low SR activity, but are well coupled and display high automaticity. For alpha and beta-myosin heavy chain (Mhc) and cardiac Troponin I and slow skeletal Troponin I, developmental transitions have been observed in differentiated ES cell cultures. Expression of Mlc2v and Anf is often used to demarcate ventricular-like and atrial-like cells in ES cell cultures, respectively, although in ESDCs, Anf expression does not exclusively identify atrial cardiomyocytes and may be a general marker of the working myocardial cells.

A “atrial progenitor” is defined as a cell that is capable (without dedifferentiation or reprogramming) of giving rise to progeny that include smooth muscle and cardiomyocytes, such as atrial progenitors.

In some embodiments, such atrial progenitors, such as Isl1⁺/SLN⁺ atrial progenitors as disclosed herein may express other markers typical of the lineage, including, without limitation, cardiac troponin I (cTnI), cardiac troponin T (cTnT), sarcomeric myosin heavy chain (MHC), GATA4, SLN, N-cadherin, beta1-adrenoreceptor (beta1-AR), ANF, the MEF-2 family of transcription factors, creatine kinase MB (CK-MB), myoglobin, or atrial natriuretic factor (ANF).

Throughout this disclosure, techniques and compositions that refer to “cardiomyocytes” or “atrial progenitors” can be taken to apply equally to cells at any stage of cardiomyocyte ontogeny without restriction, as defined above, unless otherwise specified. The cells may or may not have the ability to proliferate or exhibit contractile activity. The culture conditions may optionally comprise agents that enhance differentiation into a specific lineage, such as smooth muscle cells or atrial myocytes. For example, smooth muscle differentiation may be promoted by including cardiotrophic agents in the culture, e.g. agents capable of forming high energy phosphate bonds (such as creatine) and acyl group carrier molecules (such as carnitine); and a cardiomyocyte calcium channel modulator (such as taurine). Optionally, cardiotropic factors, including, but not limited to those described in U.S. Patent Application Serial No. 20030022367, may be added to the culture. Such factors may include, for example but not limited to nucleotide analogs that affect DNA methylation and alter expression of cardiomyocyte-related genes; TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the cripto gene; antibodies, peptidomimetics with agonist activity for the same receptors, pseudo ligands, for example peptides and antibodies, cells secreting such factors, and other methods for directed differentiation of stem cells along specific cell lineages in particular cardiomyocyte lineages.

In some embodiments, Isl1⁺/SLN⁺ atrial progenitors as disclosed herein can differentiate into cells that demonstrate spontaneous periodic contractile activity, whereas others may differentiated into cells with non-spontaneous contractile activity (evoked upon appropriate stimulation). Spontaneous contraction generally means that, when cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. Non-spontaneous contraction may be observed, for example, in the presence of pacemaker cells, or other stimulus.

### Methods for Identification of Isl1⁺/SLN⁺ Atrial Progenitors

Methods to determine the expression, for example the expression of RNA or protein expression of markers of Isl1⁺/SLN⁺ atrial progenitors as disclosed herein, such as Isl-1 and SLN expression are well known in the art, and are encompassed for use in this invention. Such methods of measuring gene expression are well known in the art, and are commonly performed on using DNA or RNA collected from a biological sample of the cells, and can be performed by a variety of techniques known in the art, including but not limited to, PCR, RT-PCR, quantitative RT-PCR (qRT-PCR), hybridization with probes, northern blot analysis, in situ hybridization, microarray analysis, RNA protection assay, SAGE or MPSS. In some embodiments, the probes used detect the nucleic acid expression of the marker genes can be nucleic acids (such as DNA or RNA) or nucleic acid analogues, for example peptide-nucleic acid (PNA), pseudocomplementary PNA (pcPNA), locked nucleic acid (LNA) or analogues or variants thereof.

In other embodiments, the expression of the markers can be detected at the level of protein expression. The detection of the presence of nucleotide gene expression of the markers, or detection of protein expression can be similarity analyzed using well known techniques in the art, for example but not limited to immunoblotting analysis, western blot analysis, immunohistochemical analysis, ELISA, and mass spectrometry. Determining the activity of the markers, and hence the presence of the markers can be also be done, typically by in vitro assays known by a person skilled in the art, for example Northern blot, RNA protection assay, microarray assay etc of downstream signaling pathways of Isl1 or SLN. In particular embodiments, qRT-PCR can be conducted as ordinary qRT-PCR or as multiplex qRT-PCR assay where the assay enables the detection of multiple markers simultaneously, for example Isl-1 and/or SLN, either together or separately from the same reaction sample.

One variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996). Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994. TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data. 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct). To minimize errors and the effect of sample-to-sample variation, RT-PCR is usually performed using an internal standard. The ideal internal standard is expressed at a relatively constant level among different tissues, and is unaffected by the experimental treatment. RNAs frequently used to normalize patterns of gene expression are mRNAs for the housekeeping genes glyceraldehyde-3-phosphate-dehydrogenase (GAPDH) and β-actin.

In some embodiments, the systems for real-time PCR uses, for example, Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the sequence of interest are generally sufficient. In addition, a standard curve is generated for the control sequence. This permits standardization of initial content of the nucleic acid of interest in a tissue sample to the amount of control for comparison purposes.

Other methods for detecting the expression of the marker gene are well known in the art and disclosed in patent application WO200004194, incorporated herein by reference. In an exemplary method, the method comprises amplifying a segment of DNA or RNA (generally after converting the RNA to cDNA) spanning one or more known isoforms of the markers (such as Isl-1, Nkx2.5, flk1) gene sequences. This amplified segment is then subjected to a detection method, such as signal detection, for example fluorescence, enzymatic etc. and/or polyacrylamide gel electrophoresis. The analysis of the PCR products by quantitative mean of the test biological sample to a control sample indicates the presence or absence of the marker gene in the cardiovascular stem cell sample. This analysis may also be performed by established methods such as quantitative RT-PCR (qRT-PCR).

The methods of RNA isolation, RNA reverse transcription (RT) to cDNA (copy DNA) and cDNA or nucleic acid amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Møller, R. Beynon, C. Howe, Springer Verlag; 1st edition (Oct. 15, 2000), ISBN: 0387916008. Other methods for detecting expression of the marker genes by analyzing RNA expression comprise methods, for example but not limited to, Northern blot, RNA protection assay, hybridization methodology and microarray assay etc. Such methods are well known in the art and are encompassed for use in this invention.

Primers specific for PCR application can be designed to recognize nucleic acid sequence encoding Isl1 and SLN, are well known in the art. For purposes of an example only, the nucleic acid sequence encoding human Isl1 can be identified by accession number: BC031213 (amino acid and nucleotide sequences disclosed as SEQ ID NOS 1 and 2, respectively) or NM_{—}002202 (amino acid and nucleotide sequences disclosed as SEQ ID NOS 1 and 3, respectively). For purposes of an example, the nucleic acid sequence encoding human SNL can be identified by accession no U96094 (amino acid and nucleotide sequences disclosed as SEQ ID NOS 4 and 5, respectively) or NM_{—}003063 (amino acid and nucleotide sequences disclosed as SEQ ID NOS 4 and 6, respectively) or Gene ID: 6588 (SEQ ID NO:7).

Any suitable immunoassay format known in the art and as described herein can be used to detect the presence of and/or quantify the amount of marker, for example Isl-1 or SLN, markers expressed by the cardiovascular stem cell. The invention provides a method of screening for the markers expressed by the Isl1⁺/SLN⁺ atrial progenitors by immunohistochemical or immunocytochemical methods, typically termed immunohistochemistry (“IHC”) and immunocytochemistry (“ICC”) techniques. IHC is the application of immunochemistry on samples of tissue, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically recognize and bind to target molecules on the inside or on the surface of cells, for example Isl-1 and/or SLN. In some embodiments, the antibody contains a reporter or marker that will catalyze a biochemical reaction, and thereby bring about a change color, upon encountering the targeted molecules. In some instances, signal amplification may be integrated into the particular protocol, wherein a secondary antibody, that includes the marker stain, follows the application of a primary specific antibody. In such embodiments, the marker is an enzyme, and a color change occurs in the presence and after catalysis of a substrate for that enzyme.

Immunohistochemical assays are known to those of skill in the art (e.g., see Jalkanen, et al., J. Cell. Biol. 101:976-985 (1985); Jalkanen, et al., J. Cell. Biol. 105:3087-3096 (1987). Antibodies, polyclonal or monoclonal, can be purchased from a variety of commercial suppliers, or may be manufactured using well-known methods, e.g., as described in Harlow et al., Antibodies: A Laboratory Manual, 2nd Ed; Cold. Spring Harbor Laboratory Press, Cold Spring Harbor, N.Y. (1988). In general, examples of antibodies useful in the present invention include anti-Islet1 or anti-SLN antibodies. Such antibodies can be purchased, for example, from Developmental Hybridoma Bank; BD PharMingen; Biomedical Technologies; Sigma; RDI; Roche and other commercially available sources. Alternatively, antibodies (monoclonal and polyclonal) can easily produced by methods known to person skilled in the art. In alternative embodiments, the antibody can be an antibody fragment, an analogue or variant of an antibody.

In some embodiments, any antibodies that recognize Isl-1 or SLN can be used by any persons skilled in the art, and from any commercial source. Examples of such antibodies include but are not limited to: anti-Isl1 (mouse monoclonal antibody, clone 39.4D5, Developmental Hybridoma bank); anti-Isl1 from Sigma, anti-Isl1 from Abcam; anti-SLN.

For detection of the makers by immunohistochemistry, the cardiovascular stem cells may be fixed by a suitable fixing agent such as alcohol, acetone, and paraformaldehyde prior to, during or after being reacted with (or probed) with an antibody. Conventional methods for immunohistochemistry are described in Harlow and Lane (Eds) (1988) In “Antibodies A Laboratory Manual”, Cold Spring Harbor Press, Cold Spring Harbor, N.Y.; Ausbel et al (Eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like. For direct labeling techniques, a labeled antibody is utilized. For indirect labeling techniques, the sample is further reacted with a labeled substance. Alternatively, immunocytochemistry may be utilized. In general, cells are obtained from a patient and fixed by a suitable fixing agent such as alcohol, acetone, and paraformaldehyde, prior to, during or after being reacted with (or probed) with an antibody. Methods of immunocytological staining of biological samples, including human samples, are known to those of skill in the art and described, for example, in Brauer et al., 2001 (FASEB J, 15, 2689-2701), Smith Swintosky et al., 1997. Immunological methods of the present invention are advantageous because they require only small quantities of biological material, such as a small quantity of cardiovascular stem cells. Such methods may be done at the cellular level and thereby necessitate a minimum of one cell.

In some embodiments, cells can be permeabilized to stain cytoplasmic molecules. In general, antibodies that specifically bind a differentially expressed polypeptide are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

In a different embodiment, antibodies (a term that encompasses all antigen-binding antibody derivatives and antigen-binding antibody fragments) that recognize the markers Isl1 or SLN are used to detect cells that express the markers. The antibodies bind at least one epitope on one or more of the markers and can be used in analytical techniques, such as by protein dot blots, sodium dodecyl sulfate polyacrylamide gel electrophoresis (SDS-PAGE), or any other gel system that separates proteins, with subsequent visualization of the marker (such as Western blots). Antibodies can also be used, for example, in gel filtration or affinity column purification, or as specific reagents in techniques such as fluorescent-activated cell sorting (FACS). Other assays for cells expressing a specific marker can include, for example, staining with dyes that have a specific reaction with a marker molecule (such as ruthenium red and extracellular matrix molecules), identification specific morphological characteristics (such as the presence of microvilli in epithelia, or the pseudopodialfilopodia in migrating cells, such as fibroblasts and mesenchyme). Biochemical assays include, for example, assaying for an enzymatic product or intermediate, or for the overall composition of a cell, such as the ratio of protein to lipid, or lipid to sugar, or even the ratio of two specific lipids to each other, or polysaccharides. If such a marker is a morphological and/or functional trait or characteristic, suitable methods including visual inspection using, for example, the unaided eye, a stereomicroscope, a dissecting microscope, a confocal microscope, or an electron microscope are encompassed for use in the invention. The invention also contemplates methods of analyzing the progressive or terminal differentiation of a cell employing a single marker, as well as any combination of molecular and/or non-molecular markers.

Various methods can be utilized for quantifying the presence of the selected markers and or reporter gene. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

Also encompassed for use in this invention, is the isolation of Isl1⁺/SLN⁺ atrial progenitors as disclosed herein by the use of an introduced reporter gene that aids with the identification of the Isl1⁺/SLN⁺ atrial progenitor cells. For example, an Isl1⁺/SLN⁺ atrial progenitor can be genetically engineered to express a construct comprising a reporter gene which can be used for selection and identification purposes. For example, the Isl1⁺/SLN⁺ atrial progenitor is genetically engineered to comprise a reporter gene, for example but not limited to a fluorescent protein, enzyme or resistance gene, which is operatively linked to a particular promoter (for example, but not limited to Isl1, and/or SLN gene). In such an embodiment, when the cell expresses the gene to which the reporter of interest is operatively linked, it also expresses the reporter gene, for example the enzyme, fluorescent protein or resistance gene. Cells that express the reporter gene can be readily detected and in some embodiments positively selected for cells comprising the reporter gene or the gene product of the reporter gene. Other reporter genes that can be used include fluorescent proteins, luciferase, alkaline phosphatase, lacZ, or CAT.

This invention also encompasses the generation of useful clonal reporter cell lines of Isl1⁺/SLN⁺ atrial progenitors of the invention that could comprise multiple reporters to help identify Isl1⁺/SLN⁺ atrial progenitors that have differentiated along particular and/or multiple lineages, such as smooth muscle or cardiomyocyte lineages. Cells expressing these reporters could be easily purified by FACS, antibody affinity capture, magnetic separation, or a combination thereof. The purified or substantially pure reporter-expressing cells can be used for genomic analysis by techniques such as microarray hybridization, SAGE, MPSS, or proteomic analysis to identify more markers that characterize the Isl1⁺/SLN⁺ atrial progenitors. These methods can be used to identify cells in an undifferentiated Isl1⁺/SLN⁺ atrial progenitor, for instance cells that have not differentiated along the desired lineages, as well as populations of cells that have differentiated along the desired lineages, such as smooth muscle cell or cardiomyocyte linages. In some embodiments, there are many cells that have not differentiated along the desired lineages; the desired cells may be isolated and subcultured to generate a substantially purified population of the desired Isl1⁺/SLN⁺ atrial progenitor. In some embodiments, where the reporter gene is a resistance gene, the resistance gene can be, for example but not limited to, genes for resistance to amplicillin, chloroamphenicol, tetracycline, puromycin, G418, blasticidin and variants and fragments thereof. In other embodiments, the reporter gene can be a fluorescent protein, for example but not limited to: green fluorescent protein (GFP); green fluorescent-like protein (GFP-like); yellow fluorescent protein (YFP); blue fluorescent protein (BFP); enhanced green fluorescent protein (EGFP); enhanced blue fluorescent protein (EBFP); cyan fluorescent protein (CFP); enhanced cyan fluorescent protein (ECFP); red fluorescent protein (dsRED); and modifications and fluorescent fragments thereof.

In some embodiments, methods to remove unwanted cells are encompassed, by removing unwanted cells by negative selection. For example, unwanted antibody-labeled cells are removed by methods known in the art, such as labeling a cell population with an antibody or a cocktail of antibodies, to a cell surface protein and separation by FACS or magnetic colloids. In an alternative embodiment, the reporter gene may be used to negatively select non-desired cells, for example a reporter gene encodes a cytotoxic protein in cells that are not desired. In such an embodiment, the reporter gene is operatively linked to a regulatory sequence of a gene normally expressed in the cells with undesirable phenotype.

One embodiment of the invention is a composition of Isl1⁺/SLN⁺ atrial progenitors as disclosed herein comprising Isl1⁺/SLN⁺ atrial progenitor positive for islet-1 and SLN. In some embodiments, the Isl1⁺/SLN⁺ atrial progenitors are of mammalian origin, and in some embodiments they are of human origin. In other embodiments, the Isl1⁺/SLN⁺ atrial progenitors are of rodent origin, for example mouse, rat or hamster, and in another embodiment, the cardiovascular stem cell is a genetically engineered stem cell. In some embodiments, the composition is substantially pure for Isl1⁺/SLN⁺ atrial progenitors.

Methods to Generate Isl1⁺/SLN⁺ Atrial Progenitors from Mature Cardiomyocytes Using Cardiac-Specific Mesenchymal Cells

Another aspect of the invention relates to methods for generating Isl1⁺/SLN⁺ atrial progenitors. In particular one embodiment of the present invention relates to methods for the generation of Isl1⁺/SLN⁺ atrial progenitors from cardiomyocytes, such as for example atrial myocytes. Accordingly, one embodiment of the present invention relates to reprogramming cardiomyocytes, such as atrial myocytes back to the Isl1⁺/SLN⁺ atrial progenitor phenotype.

In another embodiment, the present invention relates to methods for the generation of Isl1⁺/SLN⁺ atrial progenitors from Islet 1⁺ progenitors which are SLN-negative (SLN⁻). Accordingly, one embodiment of the present invention relates to differentiating islet1 progenitors to the Isl1⁺/SLN⁺ atrial progenitor phenotype.

In some embodiments, the methods of the invention provide enrichment of Isl1⁺/SLN⁺ atrial progenitors without first sorting the stem cells by positive selection methods such as FACS sorting magnetic colloid sorting or other sorting method described above. Therefore the methods of the invention do not require enrichment of Isl1⁺/SLN⁺ atrial progenitors based on prior identification of Isl1⁺/SLN⁺ atrial progenitors markers, and benefit from the absence of requiring a specific marker (either an endogenously expressed marker, and/or a genetically introduced reported gene) for enrichment. The method of the invention therefore enables enrichment of Isl1⁺/SLN⁺ atrial progenitors from either Islet1⁺ (SLN⁻) progenitors or cardiomyocytes such as atrial myocytes from any source. This has great advantages over existing methods with respect to clinical use of Isl1⁺/SLN⁺ atrial progenitors for therapeutic use, as the Isl1⁺/SLN⁺ atrial progenitors can be enriched from any subject or source for autologous stem cell transplantation without the need to genetically modify the cells for enrichment.

In this aspect of the invention, the method provides for generation of Isl1⁺/SLN⁺ atrial progenitors by culturing cardiomyocytes, such as atrial myocytes on a cardiac mesenchymal feeder layer. As described herein, the present invention provides methods for culture conditions that (i) enrich for Isl1⁺/SLN⁺ atrial progenitors, and (ii) promote proliferation without promoting differentiation of Isl1⁺/SLN⁺ atrial progenitors. Most conventional methods to isolate a particular stem cell of interest involve positive selection using markers of interest. The methods as disclosed herein provide a novel means to generate Isl1⁺/SLN⁺ atrial progenitors without the use of markers. The method for isolating and enriching Isl1⁺/SLN⁺ atrial progenitors as disclosed herein comprise culturing cardiomyocytes, such as atrial cardiomyocytes in a growth environment that enables reprogramming of the atrial cardiomyocyte back to an earlier developmental stage and to become Isl1⁺/SLN⁺ atrial progenitors. In some embodiments, the growth environment is provided by the presence of cardiac mesenchymal cells.

In one embodiment, the present invention provides methods for the generation of Isl1⁺/SLN⁺ atrial progenitors. In such an embodiment, the method encompasses culturing the cardiomyocytes, such as atrial myocytes on a cardiac mesenchymal cell (CMC) feeder layer. In some embodiments the method encompasses isolation of atrial myocytes from, for example, embryonic tissue, pre-fetal and fetal tissue, postnatal tissue, and adult tissue.

Alternatively, the Isl1⁺/SLN⁺ atrial progenitors can also be derived from Islet 1⁺ progenitors that are SLN-negative. Such Islet 1+ progenitors and methods of their isolation, identification are disclosed in U.S. Provisional Patent Applications 60/856,490 and 60/860,354 and, and International Application PCT/US07/23155, which are incorporated herein in their entirety by reference.

Without being bound to theory, feeder cell layers have conventionally been used for the continuous culturing and propagation of ES cells or stem cell lines in culture. Typical layers of feeder cells comprise fibroblasts derived from embryonic or fetal tissue, and are well known by persons skilled in the art. Recently, mesenchymal cells have been used as feeder cells for the culturing of stem cells, for example in the culturing of islet-1 positive stem cells (see Patent Application No. WO 2004/070013, which is incorporated herein in its entirety by reference). However, methods using feeder cells, in particular mesenchymal feeder cells for the enrichment and isolation of stem cells have not been described.

Typically, conventional methods to isolate a particular progenitor cell of interest involve positive and negative selection using markers of interest. For example, agents can be used to recognize markers present on the Isl1⁺/SLN⁺ atrial progenitors, for instance labeled antibodies that recognize and bind to cell-surface markers or antigens on the Isl1⁺/SLN⁺ atrial progenitors which can be used to separate and isolate the Isl1⁺/SLN⁺ atrial progenitors using fluorescent activated cell sorting (FACS), panning methods, magnetic particle selection, particle sorter selection and other methods known to persons skilled in the art, including density separation (Xu et al. (2002) Circ. Res. 91:501; U.S. patent application Ser. No. 20030022367) and separation based on other physical properties (Doevendans et al. (2000) J. Mol. Cell. Cardiol. 32:839-851). Alternatively, genetic selection methods can be used, where an Isl1⁺/SLN⁺ atrial progenitors can be genetically engineered to express a reporter protein operatively linked to a tissue-specific promoter and/or a specific gene promoter, therefore the expression of the reporter can be used for positive selection methods to isolate and enrich the Isl1⁺/SLN⁺ atrial progenitors. For example, a fluorescent reporter protein can be expressed in the desired stem cell by genetic engineering methods to operatively link the marker protein to the promoter expressed in a desired stem cell (Klug et al. (1996) J. Clin. Invest. 98:216-224; U.S. Pat. No. 6,737,054). Other means of positive selection include drug selection, for instance such as described by Klug et al, supra, involving enrichment of desired cells by density gradient centrifugation. Negative selection can be performed and selecting and removing cells with undesired markers or characteristics, for example fibroblast markers, epithelial cell markers etc.

In some embodiments, the methods as disclosed herein comprise plating embryonic or postnatal cardiomyocytes such as atrial myocytes, or Isl1⁺ progenitors (which are SLN−) on a feeder layer of mesenchymal cells such as cardiac messenchymal feeder layer. In one embodiment, the cardiomyocytes, such as atrial myocytes, or Isl1⁺ progenitors are plated as single cells. In another embodiment, the cardiomyocytes such as atrial myocytes are plated as aggregates of cells, for example the atrial myocytes are present in a tissue, for example the tissue can be embryonic tissue, fetal tissue, pre-fetal tissue, neonatal tissue, post-natal tissue or adult tissue. In some embodiments, when cultured in the presence of cardiac messenchymal feeder layer cells, the cardiomyocytes such as atrial myocytes reprogram to an earlier developmental stage to become Isl1⁺/SLN⁺ atrial progenitors.

In embodiments where Isl1⁺/SLN⁺ atrial progenitors are generated from Isl1⁺ progenitors which are SLN-negative, the source of Isl1⁺ progenitors can be obtained from by methods commonly known in the art, such as for example, as disclosed in Provisional Patent Applications 60/856,490 and 60/860,354 and International Application PCT/US07/23155, which are incorporated herein in their entirety by reference. Accordingly, the Isl1⁺/SLN⁺ atrial progenitors can be generated by culturing the Is1⁺ progenitors in the presence of a cardiac messenchymal feeder layer, wherein the Is1⁺ progenitors are origionally derived from various sources, such as, for example but not limited to embryonic stem (ES) cells, adult stem cells (ASC), embryoid body's (EB).

In some embodiments, the cardiomyocytes, such as atrial myocytes can be in the presence of the cardiac mesenchymal cell feeder layer, for example the cardiomyocytes, such as atrial myocytes can be cultured on a layer suspended above or below the cardiac mesenchymal feeder layer. In an alternative embodiment, the cardiomyocytes, such as atrial myocytes may be in contact with and/or grow on the same surface of the cardiac mesenchymal cells. In an alternative embodiment, the cardiomyocytes, such as atrial myocytes are grown in a culture with the cardiac mesenchymal cells in any form whereby the cardiac mesenchymal cells provide an environment whereby the signals from the cardiac mesenchymal cells cause the cardiomyocytes, such as atrial myocytes to reprogram to become Isl1⁺/SNL⁺ atrial progenitors. As a non-limiting example, where the signals from the cardiac mesenchymal cells cause the cardiomyocytes, such as atrial myocytes to reprogram and enter an earlier developmental stage such as the Isl1⁺/SNL⁺ atrial progenitor state.

In some embodiments, the mesenchymal cells are from cardiac tissue. In some embodiments, the cardiac mesenchymal cells are from embryonic tissue, fetal tissue, pre-fetal tissue, adult tissue. In some embodiments, the cardiac mesenchymal cells are from the same species origin as the species origin of the cardiomyocytes, such as atrial myocytes. In alternative embodiments, the cardiac mesenchymal cells are from a different species as the species of the cardiomyocytes, such as atrial myocytes. In some embodiments, the cardiac mesenchymal cells have been genetically modified, and in some embodiments, the cardiac mesenchymal cells are from genetically engineered or transgenic organisms. In some embodiments, the cardiomyocytes, such as atrial myocytes are genetically engineered cardiomyocytes, such as atrial myocytes.

In one embodiment of the invention, the cardiomyocytes, such as atrial myocytes cultured with cardiac mesenchymal cells can be optionally selected. In some embodiments, the selection method uses markers expressed by reprogrammed cardiomyocytes, or reprogrammed atrial myocytes, such as markers for Isl1 and/or SLN. In some embodiments, such selection methods can also be combined with other enrichment methods, including genetic selection (Klug et al. (1996) J. Clin. Invest. 98:216-224; U.S. Pat. No. 6,737,054); density separation (Xu et al. (2002) Circ. Res. 91:501; U.S. patent application Ser. No. 20030022367); separation based on physical properties (Doevendans et al. (2000) J. Mol. Cell. Cardiol. 32:839-851); and the like. These references are herein specifically incorporated by reference for methods of enriching for cardiomyocyte-derived Isl1⁺/SNL⁺ atrial progenitor, but the methods can be applied to methods for enriching for Isl1⁺ progenitor (SLN⁻) derived Isl1⁺/SNL⁺ atrial progenitors. Markers for selection include, without limitation, biomolecules present on the cell surface. Such markers include markers for positive selection, which are present on the stem cells of interest, or markers for negative selection, which are absent on the stem cells of interest, but which typically are present on the undesired cells, for example cells such as cardiomyocytes etc.

### Differentiation of Isl1+/SLN+ Atrial Progenitors Along Cardiomyocyte Lineages

Another embodiment of the present invention relates to the production of large numbers of cardiomyocytes. In some embodiments, the present invention relates to the production of large numbers of cardiomyocytes from a subject. In such an embodiment, cardiomyocytes from the subject can be used to generate cardiomyocyte-derived Isl1⁺/SNL⁺ atrial progenitors by the methods as disclosed herein, and such Isl1⁺/SNL⁺ atrial progenitor can be subsequently differentiated to becomes smooth muscle and/or cardiomyocytes such as atrial myocytes by the methods as disclosed herein. Thus, the present invention relates to methods to produce cardiomyocyte-derived Isl1⁺/SNL⁺ atrial progenitors from somatic stem cells, and then, subsequently the cardiomyocyte-derived Isl1⁺/SNL⁺ atrial progenitors can be differentiated to produce cardiomyocytes in large numbers. Hence, in some embodiments the present invention is highly useful for producing useful quantities of cardiomyocytes by reprogramming cardiomyocytes to an earlier developmental stage, propagating them and inducing their differentiation along cardiomyocyte and smooth muscle phenotypes.

In some embodiments, the Isl1⁺/SNL⁺ atrial progenitors, such as cardiomyocyte-derived Isl1⁺/SNL⁺ atrial progenitors can be differentiated along cardiomyocyte lineages. Without wishing to be bound to theory, during normal cardiac morphogenesis, the cranio-lateral part of the visceral mesoderm becomes committed to the cardiogenic lineage. Several heart-associated transcription factors, such as Nkx2.5, Hand1, 2, Srf, Tbx5, Gata4, 5, 6 and Mef2c, become expressed in the cardiogenic region. The first possible overt sign of restriction of gastrulating mesodermal cells to the cardiogenic lineage is the expression of the basic helix-loop-helix transcription factor Mesp1. Cardiogenic mesoderm expressing Mesp1 is pluripotent and contains the precursors for the endocardial/endothelial, the epicardial and the myocardial lineages. The cardiomyocytes of the primary heart tube are characterized by low abundance of sarcomeric and sarcoplasmatic reticular transcripts. Myosin light chain (Mlc) 2v is expressed in a part of the tube that gives rise not only to ventricular chamber myocardium, but also to parts of the atrial chambers and to the atrioventricular node. alpha and beta-myosin heavy chain (Mhc), Mc1a, 1v and 2a are initially expressed in the entire heart-tube in gradients, and are later restricted to their compartments.

A number of well-known cardiomyocyte markers are well known by persons of ordinary skill in the art and can be used for positive selection of Isl1⁺/SNL⁺ atrial progenitors that have differentiated along cardiomyocyte lineages. In some embodiments, useful markers for positive selection of cardiomyocytes may include, without limitation, one, two or more of NCAM (CD56); HNK-1; L-type calcium channels; cardiac sodium-calcium exchanger; etc. Additional cytoplasmic markers for cardiomyocyte subsets are also of interest, e.g. Mlc2v for ventricular-like working cells; and Anf as a general marker of the working myocardial cells. Markers for pacemaker cells also include HCN2, HCN4, connexin 40, etc.

Alternatively, negative selection of Isl1⁺/SNL⁺ atrial progenitors that express markers indicative of an undesired cell types and/or differentiation along undesired lineages is also encompassed in the methods as disclosed herein. For example, negative selection of Isl1⁺/SNL⁺ atrial progenitors can be used to exclude Isl1⁺/SNL⁺ atrial progenitors which express markers with unwanted characteristics, for example markers expressed on fibroblasts, epithelial cells, etc. Epithelial cells may be selected for as ApCAM positive. A fibroblast specific selection agent is commercially available from Miltenyi Biotec (see Fearns and Dowdle (1992) Int. J. Cancer 50:621-627 for discussion of the antigen). Markers found on ES cells suitable for negative selection include SSEA-3, SSEA-4, TRA-I-60, TRA-1-81, and alkaline phosphatase.

Screening for Agents that Promote Reprogramming of Cardiomyocytes

Another aspect of the invention relates to methods to screen for agents, for example chemicals molecules and gene products that promote, for example the reprogramming of cardiomyocytes such as atrial myocytes into Isl1⁺/SNL⁺ atrial progenitors.

In another embodiment, the methods as disclosed herein provide an assay to identify agents which are toxic to Isl1⁺/SNL⁺ atrial progenitors. In some embodiments, the agents, drugs and/or compounds can be existing drugs or compounds, and in other embodiments, the drugs or compounds can be new or modified drugs, compounds or variants thereof. In another embodiment, the methods as disclosed herein permits the screening of agents that affect (i.e. promote or inhibit) Isl1⁺/SNL⁺ atrial progenitor differentiation into cardiomyocyte lineages. In some embodiments, the Isl1⁺/SNL⁺ atrial progenitor can be a cardiomyocyte-derived Isl1⁺/SNL⁺ atrial progenitor or a Isl1+ progenitor derived Isl1⁺/SNL⁺ atrial progenitor, and can also include, for example but not limited to a genetic variant and/or a genetically modified Isl1⁺/SNL⁺ atrial progenitors.

In some embodiments, the methods as disclosed herein related to culturing cardiomyocytes in the presence of agents, such as in vitro assays, and identifying agents that promote the reprogramming of cardiomyocytes into Isl1⁺/SNL⁺ atrial progenitor. In alternative embodiments, the methods as disclosed herein provide methods for the identifying agents which affect the differentiation of Isl1⁺/SNL⁺ atrial progenitor, including differentiation of Isl1⁺/SNL⁺ atrial progenitor along the cardiomyocyte lineages. Of particular interest are screening assays for agents that are active on human Isl1⁺/SNL⁺ atrial progenitor, such as human cardiomyocyte-derived Isl1⁺/SNL⁺ atrial progenitors. A wide variety of assays may be used for this purpose, including immunoassays for protein binding; determination of cell growth, differentiation and functional activity; production of factors; and the like.

Alternatively, the methods are useful in screening for agents to promote the differentiation of Isl1+ progenitors (that are SLN−) to differentiate into Isl1⁺/SNL⁺ atrial progenitors.

In some embodiments, for identification of agents which promote the reprogramming of cardiomyocytes, such as atrial myocytes into Isl1⁺/SNL⁺ atrial progenitor, the cardiomyocytes are contacted with the agent of interest, and the effect of the agent assessed by monitoring output parameters, such as expression of markers such as increase expression of Isl1⁺ and/or SLN⁺ and loss of expression of cardiomyocyte markers, increased cell viability, differentiation characteristics, multipotenticy capacity and the like. In some embodiments, the cardiomyocytes may be freshly isolated, cultured, genetically engineered as described above, or the like. The cardiomyocytes can be environmentally induced variants of clonal cultures: e.g. split into independent cultures and grown under distinct conditions, for example with or without virus; in the presence or absence of other cytokines or combinations thereof.

Alternatively, the cardiomyocytes can be variants with a desired pathological characteristic. For example, the desired pathological characteristic includes a mutation and/or polymorphism which contribute to a disease pathology, such as a cardiovascular disease pathology as disclosed herein. In such an embodiment, the methods as disclosed herein can be used to screen for agents which alleviate the pathology. In alternative embodiments, the methods as disclosed herein can be used to screen for agents which affect Isl1⁺/SNL⁺ atrial progenitors and/or cardiomyocytes which comprise particular mutations and/or polymorphisms differently as compared with wild-type Isl1⁺/SNL⁺ atrial progenitors and/or cardiomyocytes (i.e. Isl1⁺/SNL⁺ atrial progenitors or cardiomyocytes without the mutation and/or polymorphism). Therefore, the methods as disclosed herein can be used for example, to assess an effect of a particular drug and/or agent on Isl1⁺/SNL⁺ atrial progenitors and/or cardiomyocytes from a defined subpopulation of people and/or cells, therefore acting as a high-throughput screen for personalized medicine and/or pharmogenetics. The manner in which cells respond to an agent, particularly a pharmacologic agent, including the timing of responses, is an important reflection of the physiologic state of the cell.

In some embodiments, agents used in the screening methods as disclosed herein can be selected from a group of a chemical, small molecule, chemical entity, nucleic acid sequences, an action; nucleic acid analogues or protein or polypeptide or analogue of fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof. The agent may be applied to the media, where it contacts the cell (such as Isl1⁺/SNL⁺ atrial progenitor and/or cardiomyocyte) and induces its effects. Alternatively, the agent may be intracellular within the cell (i.e. expressed by the Isl1⁺/SNL⁺ atrial progenitor) as a result of introduction of the nucleic acid sequence into the Isl1⁺/SNL⁺ atrial progenitor and its transcription resulting in the production of the nucleic acid and/or protein agent within the cell. An agent also encompasses any action and/or event the cells are subjected to. As a non-limiting examples, an action can comprise any action that triggers a physiological change in the cell, for example but not limited to; heat-shock, ionizing irradiation, cold-shock, electrical impulse, light and/or wavelength exposure, UV exposure, pressure, stretching action, increased and/or decreased oxygen exposure, exposure to reactive oxygen species (ROS), ischemic conditions, fluorescence exposure etc. Environmental stimuli also include intrinsic environmental stimuli defined below. The exposure to agent may be continuous or non-continuous.

The term “agent” refers to any chemical, entity or moiety, including without limitation synthetic and naturally-occurring non-proteinaceous entities. In certain embodiments the compound of interest is a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

In some embodiments, the agent is an agent of interest including known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidate agents also include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as agents are pharmacologically active drugs, genetically active molecules, etc. Compounds of interest include, for example, chemotherapeutic agents, hormones or hormone antagonists, growth factors or recombinant growth factors and fragments and variants thereof. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for effect on the stem cell by adding the agent to at least one and usually a plurality of stem cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method. In some embodiments, agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Optionally, the cardiomyocyte and/or Isl1⁺/SNL⁺ atrial progenitor used in the screening assays can be manipulated to express desired gene products. Gene therapy can be used to either modify a cell to replace a gene product or add or knockdown a gene product. In some embodiments the genetic engineering is done to facilitate regeneration of tissue, to treat disease, or to improve survival of the cells following implantation into a subject (i.e. prevent rejection). Alternatively, in some embodiments the cardiomyocyte and/or Isl1⁺/SNL⁺ atrial progenitor can be genetically engineered prior to their use in the assay, or alternatively, the cardiomyocyte and/or Isl1⁺/SNL⁺ atrial progenitor can be transfected while they are being assessed for an effect of the agent on the reprogramming of the cardiomyocyte to a Isl1⁺/SNL⁺ atrial progenitor, or the effect of the agent on the differentiation of Isl1⁺/SNL⁺ atrial progenitors along cardiac lineages. Techniques for transfecting cells are known in the art.

A skilled artisan could envision a multitude of genes which would convey beneficial properties to the cardiomyocyte or to the cardiomyocyte-derived Isl1⁺/SNL⁺ atrial progenitor, particularly if the Isl1⁺/SNL⁺ atrial progenitor is from a subject and if such Isl1⁺/SNL⁺ atrial progenitor is to be used in transplantation (discussed in more detail below). The added gene may ultimately remain in the recipient Isl1⁺/SNL⁺ atrial progenitors and all its progeny, or may only remain transiently, depending on the embodiment. For example, genes encoding angiogenic factors could be transiently transfected into Isl1⁺/SNL⁺ atrial progenitors to promote subsequent differentiation along cardiomyocyte lineages, such as smooth muscle cells lineages. Such genes would be useful for inducing collateral blood vessel formation as the smooth muscle tissue is regenerated. It some situations, it may be desirable to transfect an Isl1⁺/SNL⁺ atrial progenitor with more than one gene.

In some instances, it is desirable to have the agent which is gene product secreted. In such cases, the gene product preferably contains a secretory signal sequence that facilitates secretion of the protein. For example, if the desired gene product is an angiogenic protein, a skilled artisan could either select an angiogenic protein with a native signal sequence, e.g. VEGF, or can modify the gene product to contain such a sequence using routine genetic manipulation (See Nabel et al., 1993).

In some embodiments, the desired gene can be transfected into the cell using a variety of techniques. Preferably, the gene is transfected into the cell using an expression vector. Suitable expression vectors include plasmid vectors (such as those available from Stratagene, Madison Wis.), viral vectors (such as replication defective retroviral vectors, herpes virus, adenovirus, adeno-virus associated virus, and lentivirus), and non-viral vectors (such as liposomes or receptor ligands).

In some embodiments, the desired gene is usually operably linked to its own promoter or to a foreign promoter which, in either case, mediates transcription of the gene product. Promoters are chosen based on their ability to drive expression in restricted or in general tissue types, for example in mesenchymal cells, or on the level of expression they promote, or how they respond to added chemicals, drugs or hormones. Other genetic regulatory sequences that alter expression of a gene may be co-transfected. In some embodiments, the host cell DNA may provide the promoter and/or additional regulatory sequences. Other elements that can enhance expression can also be included such as an enhancer or a system that results in high levels of expression.

Methods of targeting genes in mammalian cells are well known to those of skill in the art (U.S. Pat. Nos. 5,830,698; 5,789,215; 5,721,367 and 5,612,205). By “targeting genes” it is meant that the entire or a portion of a gene residing in the chromosome of a cell is replaced by a heterologous nucleotide fragment. The fragment may contain primarily the targeted gene sequence with specific mutations to the gene or may contain a second gene. The second gene may be operably linked to a promoter or may be dependent for transcription on a promoter contained within the genome of the cell. In a preferred embodiment, the second gene confers resistance to a compound that is toxic to cells lacking the gene. Such genes are typically referred to as antibiotic-resistance genes. Cells containing the gene may then be selected for by culturing the cells in the presence of the toxic compound.

Methods of gene targeting in mammals are commonly used in transgenic “knockout” mice (U.S. Pat. Nos. 5,616,491; 5,614,396). These techniques take advantage of the ability of mouse embryonic stem cells to promote homologous recombination, an event that is rare in differentiated mammalian cells. Recent advances in human embryonic stem cell culture may provide a needed component to applying the technology to human systems (Thomson; 1998). Furthermore, the methods of the present invention can be used to isolate and enrich for stem cells or progenitor cells that are capable of homologous recombination and, therefore, subject to gene targeting technology. Indeed, the ability to isolate and grow somatic stem cells and progenitor cells has been viewed as impeding progress in human gene targeting (Yanez & Porter, 1998).

### Uses of Isl1⁺/SNL⁺ Atrial Progenitors

In another aspect of the invention relates to methods to use Isl1⁺/SNL⁺ atrial progenitors in cell replacement therapy. As disclosed in the Examples (see FIG. 9 demonstrating engraftment of atrial myocyte-derived Isl1⁺/SNL⁺ atrial progenitors into ventricular wall), one embodiment of the present invention relates to the use of Isl1⁺/SNL⁺ atrial progenitors for the production of a pharmaceutical composition which can be used for transplantation into subjects in need of cardiac tissue transplantation, for example but not limited to subjects with congenital and acquired heart disease and subjects with vascular diseases. In one embodiment, the Isl1⁺/SNL⁺ atrial progenitors may be genetically modified. In another aspect, the subject may have or be at risk of heart disease and/or vascular disease. In some embodiments, the Isl1⁺/SNL⁺ atrial progenitors may be autologous and/or allogenic. In some embodiments, the subject is a mammal, and in other embodiments the mammal is a human.

The use of the Isl1⁺/SNL⁺ atrial progenitors as disclosed herein provides advantages over existing methods because the Isl1⁺/SNL⁺ atrial progenitors are already primed to differentiate along cardiomyocyte lineages. This is highly advantageous as it provides a renewable source of cardiac muscle cells derived from a subject for cell transplantation therapy in the same, or a different subject from which the cells were derived from. In particular, the methods as disclosed herein enable the production of a renewable source of cardiomyocytes from a individual subject, for example by reprogramming the cardiomyocytes (such as atrial myocytes) from the subject to become Isl1⁺/SNL⁺ atrial progenitors which can be expanded and renewed, and used, prior to or after differentiation into cardiomyocytes, for cell based therapy. In some embodiments, the Isl1⁺/SNL⁺ atrial progenitors are a renewable source of homogeneous cardiac myocytes derived from that subject which have a restricted differentiation potential to become cardiomyocytes, allowing for regeneration of specific heart structures without the risks and limitations of other cardiovascular progenitor or ES cell based systems, such as risk of teratomas (Lafamme and Murry, 2005, Murry et al, 2005; Rubart and Field, 2006) or development of other heart structures when cardiac muscle is required.

In another embodiment, the Isl1⁺/SNL⁺ atrial progenitors can be used as models for studying differentiation pathways of cardiomyocytes such as into multiple cardiomyocyte lineages, for example but not limited to, cardiac muscle cells or smooth muscle cells. In some embodiments, the Isl1⁺/SNL⁺ atrial progenitors may be genetically engineered to comprise markers operatively linked to promoters that are expressed in one or more of the lineages being studied. In some embodiments, the Isl1⁺/SNL⁺ atrial progenitors can be used as a model for studying the differentiation pathway of their into subpopulations of cardiomyocytes. In some embodiments, the Isl1⁺/SNL⁺ atrial progenitors may be genetically engineered to comprise markers operatively linked to promoters that drive gene transcription in specific cardiomyocyte subpopulations, for example but not limited to atrial, ventricular, outflow tract and conduction systems.

In some embodiments, the Isl1⁺/SNL⁺ atrial progenitors can be derived from cardiomyocytes such as atrial myocytes from a normal heart or from a disease heart. In some embodiments the disease heart carries a mutation and/or polymorphism, and in other embodiments, the disease heart has been genetically engineered to carry a mutation and/or polymorphism. In other embodiments, a Isl1⁺/SNL⁺ atrial progenitors can be derived from tissue, for example but not limited to embryonic heart, fetal heart, postnatal heart and adult heart.

In one embodiment of the invention relates to a method of treating a circulatory disorder comprising administering an effective amount of a composition comprising Isl1⁺/SNL⁺ atrial progenitors to a subject with a circulatory disorder. In a further embodiment, the invention provides a method for treating myocardial infarction, comprising administering a composition comprising Isl1⁺/SNL⁺ atrial progenitors to a subject having a myocardial infarction in an effective amount sufficient to produce cardiac muscle cells in the heart of the subject, wherein the Isl1⁺/SNL⁺ atrial progenitors differentiate into smooth muscle cells, cardiac muscle cells and cardiomyocytes. In some embodiments, the methods as disclosed herein further encompasses differentiating Isl1⁺/SNL⁺ atrial progenitors into cardiomyocytes and/or smooth muscle cells and comprising administering an effective amount of a the cardiomyocytes and/or smooth muscle cells to a subject in need of treatment.

In some embodiments, the methods as disclosed herein further provides a method of treating an injured tissue in a subject comprising: (a) determining a site of tissue injury in the subject; and (b) administering Isl1⁺/SNL⁺ atrial progenitors as disclosed herein in a composition into and around the site of tissue injury, wherein the Isl1⁺/SNL⁺ atrial progenitor composition comprises a cell that have the potential to differentiate into cardiomyocytes or smooth muscle cells after administration. In one embodiment, the site of tissue injury is injury to cardiac muscle. In a further embodiment, the tissue injury is a myocardial infarction, cardiomyopathy or congenital heart disease

In some embodiments, the Isl1⁺/SNL⁺ atrial progenitors are cardiomyocyte-derived Isl1⁺/SNL⁺ atrial progenitors. In some embodiments, the cardiomyocyte-derived Isl1⁺/SNL⁺ atrial progenitors are derived from cardiomyocytes harvested from the subject to which the cardiomyocyte-derived Isl1⁺/SNL⁺ atrial progenitors are to be administered, and as such they are an autologous source of cardiomyocyte-derived Isl1⁺/SNL⁺ atrial progenitors.

In one embodiment of the above methods, the subject is a human and the Isl1⁺/SNL⁺ atrial progenitors are human cells. In alternative embodiments, the Isl1⁺/SNL⁺ atrial progenitors can be use to treat circulatory disorder is selected from the group consisting of cardiomyopathy, myocardial infarction, and congenital heart disease. In some embodiments, the circulatory disorder is a myocardial infarction. The methods as disclosed herein provides that the differentiation of Isl1⁺/SNL⁺ atrial progenitors into cardiomyocytes such as smooth muscle cells and atrial myocytes can be used to treat myocardial infarction by reducing the size of the myocardial infarct. It is also contemplated that the differentiation of Isl1⁺/SNL⁺ atrial progenitors into cardiomyocytes can be used to treat myocardial infarction by reducing the size of the scar resulting from the myocardial infarct. The methods as disclosed herein also encompasses that Isl1⁺/SNL⁺ atrial progenitors are administered directly to heart tissue of a subject, or is administered systemically. As demonstrated in FIG. 9 in the Examples, Isl1⁺/SNL⁺ atrial progenitors can be administered ventricular wall of the heart.

In some embodiments, the methods as disclosed herein can be used to treat circulatory damage in the heart or peripheral vasculature which occurs as a consequence of genetic defect, physical injury, environmental insult or damage from a stroke, heart attack or cardiovascular disease (most often due to ischemia) in a subject, the method comprising administering (including transplanting), an effective number or amount of Isl1⁺/SNL⁺ atrial progenitors and/or their progeny (such as smooth muscle cells or cardiomyocytes as a result of the differentiation of Isl1⁺/SNL⁺ atrial progenitors) to a subject. Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

In some embodiments, the effects of Isl1⁺/SNL⁺ atrial progenitor cell delivery therapy would be demonstrated by, but not limited to, one of the following clinical measures: increased heart ejection fraction, decreased rate of heart failure, decreased infarct size, decreased associated morbidity (pulmonary edema, renal failure, arrhythmias) improved exercise tolerance or other quality of life measures, and decreased mortality. The effects of cellular therapy of Isl1⁺/SNL⁺ atrial progenitors can be evident over the course of days to weeks after the procedure. However, beneficial effects may be observed as early as several hours after the procedure, and may persist for several years.

In some embodiments, smooth muscle cells and/or cardiomyocytes which have differentiated from Isl1⁺/SNL⁺ atrial progenitors can be used for tissue reconstitution or regeneration in a human subject or other subject in need of such treatment. In some embodiments, Isl1⁺/SNL⁺ atrial progenitors and/or their progeny (such as smooth muscle cells or cardiomyocytes as a result of the differentiation of Isl1⁺/SNL⁺ atrial progenitors) are administered in a manner that permits them to graft or migrate to the intended tissue site and reconstitute or regenerate the functionally deficient area. Special devices are available that are adapted for administering cells capable of reconstituting cardiac function directly to the chambers of the heart, the pericardium, or the interior of the cardiac muscle at the desired location. In some embodiments, the Isl1⁺/SNL⁺ atrial progenitors and/or their progeny (such as smooth muscle cells or cardiomyocytes as a result of the differentiation of Isl1⁺/SNL⁺ atrial progenitors) can be administered to a recipient heart by intracoronary injection, e.g. into the coronary circulation. The Isl1⁺/SNL⁺ atrial progenitors and/or their progeny (such as smooth muscle cells or cardiomyocytes as a result of the differentiation of Isl1⁺/SNL⁺ atrial progenitors) can also be administered by intramuscular injection into the wall of the heart.

In some embodiments, the composition comprising Isl1⁺/SNL⁺ atrial progenitors is enriched for the desired smooth muscle or cardiomyocyte lineages. Usually at least about 50% of the aggregates will comprise at least one of the selected differentiating cells, more usually at least about 75% of the aggregates, and preferably at least about 90% of the aggregates. Aggregates tend to comprise similar cells, and usually at least about 50% of the total cells in the population will be the selected differentiating cells, more usually at least about 75% of the cells, and preferably at least about 90% of the cells.

The compositions as disclosed herein can have a variety of uses in clinical therapy, research, development, and commercial purposes. For therapeutic purposes, for example, Isl1⁺/SNL⁺ atrial progenitors and/or their progeny (such as smooth muscle cells or cardiomyocytes as a result of the differentiation of Isl1⁺/SNL⁺ atrial progenitors) can be administered to enhance tissue maintenance or repair of cardiac muscle for any perceived need, such as an inborn error in metabolic function, the effect of a disease condition, or the result of significant trauma. The Isl1⁺/SNL⁺ atrial progenitors and/or their progeny (such as smooth muscle cells or cardiomyocytes as a result of the differentiation of Isl1⁺/SNL⁺ atrial progenitors) that are administered to the subject not only help restore function to damaged or otherwise unhealthy tissues, but also facilitate remodeling of the damaged tissues.

To determine the suitability of cell compositions for therapeutic administration, the Isl1⁺/SNL⁺ atrial progenitors and/or their progeny (such as smooth muscle cells or cardiomyocytes as a result of the differentiation of Isl1⁺/SNL⁺ atrial progenitors) can first be tested in a suitable animal model. At one level, Isl1⁺/SNL⁺ atrial progenitors and/or their progeny (such as smooth muscle cells or cardiomyocytes as a result of the differentiation of Isl1⁺/SNL⁺ atrial progenitors) are assessed for their ability to survive and maintain their phenotype in vivo. In some embodiments, the cell compositions as disclosed herein can be administered to immunodeficient animals (such as nude mice, or animals rendered immunodeficient chemically or by irradiation). Tissues are harvested after a period of regrowth, and assessed as to whether the administered cells or progeny thereof are still present. This can be performed by administering Isl1⁺/SNL⁺ atrial progenitors and/or their progeny that express a detectable label (such as green fluorescent protein, or beta-galactosidase); that have been prelabeled (for example, with BrdU or [3H] thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

In embodiments where Isl1⁺/SNL⁺ atrial progenitors are used, or cardiomyocytes which are derived from the differentiation of Isl1⁺/SNL⁺ atrial progenitors are used, the suitability of the Isl1⁺/SNL⁺ atrial progenitor or their progeny can also be determined in an animal model by assessing the degree of cardiac recuperation that ensues from treatment with the cells of the invention. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N2 on the anterior wall of the left ventricle for approximately 20 min (Chiu et al., Ann. Thorac. Surg. 60:12, 1995). Infarction can be induced by ligating the left main coronary artery (Li et al., J. Clin. Invest. 100:1991, 1997). Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, and rate of pressure decay.

In some embodiments, Isl1⁺/SNL⁺ atrial progenitors or their progeny as disclosed herein may be administered in any physiologically acceptable excipients. The cells may be introduced by injection, catheter, or the like. In some embodiments, the Isl1⁺/SNL⁺ atrial progenitors or their progeny can be frozen at liquid nitrogen temperatures and stored for long periods of time, being capable of use on thawing. If frozen, the cells will usually be stored in a 10% DMSO, 50% FCS, 40% RPMI 1640 medium. Once thawed, the Isl1⁺/SNL⁺ atrial progenitors can be expanded, and optionally differentiated into cardiomyocytes or smooth muscle cells by the methods as disclosed herein prior to administration to the subject.

The Isl1⁺/SNL⁺ atrial progenitors or their progeny as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition as disclosed herein can also comprise or be accompanied with one or more other ingredients that facilitate the engraftment or functional mobilization of the cells. Suitable ingredients include matrix proteins that support or promote adhesion of the cells, or complementary cell types, especially endothelial cells. In another embodiment, the composition may comprise resorbable or biodegradable matrix scaffolds.

In some embodiments, the Isl1⁺/SNL⁺ atrial progenitors or their progeny as disclosed herein can be genetically altered in order to introduce genes useful in the differentiated cell, e.g. repair of a genetic defect in an individual, selectable marker, etc., or genes useful in selection against undifferentiated ES cells. Isl1⁺/SNL⁺ atrial progenitors or their progeny as disclosed herein can also be genetically modified to enhance survival, control proliferation, and the like. Isl1⁺/SNL⁺ atrial progenitors or their progeny as disclosed herein can be genetically altering by transfection or transduction with a suitable vector, homologous recombination, or other appropriate technique, so that they express a gene of interest. In one embodiment, Isl1⁺/SNL⁺ atrial progenitors or their progeny can be transfected with genes encoding a telomerase catalytic component (TERT), typically under a heterologous promoter that increases telomerase expression beyond what occurs under the endogenous promoter, (see International Patent Application WO 98/14592).

In other embodiments, a selectable marker is introduced, for example, but not limited to, to provide identification of the transplanted cells, to track the fate of the transplanted cells, for identification of which type of cell (i.e. smooth muscle cell or cardiomyocyte) the transplanted cell has differentiated into, and for use to increase the purity of the Isl1⁺/SNL⁺ atrial progenitors or their progeny. Isl1⁺/SNL⁺ atrial progenitors or their progeny can be genetically altered using vector over a 8-16 h period, and then exchanged into growth medium for 1-2 days. Genetically altered Isl1⁺/SNL⁺ atrial progenitors or their progeny can be selected using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (i.e. prevent rejection).

In an alternative embodiment, the Isl1⁺/SNL⁺ atrial progenitors or their progeny as disclosed herein can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the Isl1⁺/SNL⁺ atrial progenitors or their progeny such as smooth muscle cell or cardiomyocyte. Of particular interest are Isl1⁺/SNL⁺ atrial progenitors or their progeny that are genetically altered to express one or more growth factors of various types, cardiotropic factors such as atrial natriuretic factor, cripto, and cardiac transcription regulation factors, such as GATA-4, Nkx2.5, and Mef2-C.

Many vectors useful for transferring exogenous genes into target mammalian cells are available. The vectors may be episomal, e.g. plasmids, virus derived vectors such as cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells. In some embodiments, the vectors may include genes that must later be removed, e.g. using a recombinase system such as Cre/Lox, or the cells that express them destroyed, e.g. by including genes that allow selective toxicity such as herpesvirus TK, Bcl-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

Another aspect of the present invention relates to the administration of the Isl1⁺/SNL⁺ atrial progenitors or their progeny as disclosed herein either systemically or to a target anatomical site. The Isl1⁺/SNL⁺ atrial progenitors or their progeny (such as smooth muscle cells and/or cardiomyocytes which are derived from the differentiation of Isl1⁺/SNL⁺ atrial progenitors) can be grafted into or nearby a subject's heart, for example, or may be administered systemically, such as, but not limited to, intra-arterial or intravenous administration. In alternative embodiments, the Isl1⁺/SNL⁺ atrial progenitors or their progeny as disclosed herein can be administered in various ways as would be appropriate to implant in the cardiovascular system, including but not limited to parenteral, including intravenous and intraarterial administration, intrathecal administration, intraventricular administration, intraparenchymal, intracranial, intracisternal, intrastriatal, and intranigral administration. Optionally, the cardiovascular stem cells are administered in conjunction with an immunosuppressive agent.

The Isl1⁺/SNL⁺ atrial progenitors or their progeny as disclosed herein can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is defined in the definitions sections and is determined by such considerations as are known in the art. The amount must be effective to halt the disease progression and/or to achieve improvement, including but not limited to improved survival rate or more rapid recovery, or improvement or elimination of symptoms and other indicators as are selected as appropriate measures by those skilled in the art. Isl1⁺/SNL⁺ atrial progenitors or their progeny administration to a subject can take place but is not limited to the following locations: clinic, clinical office, emergency department, hospital ward, intensive care unit, operating room, catheterization suites, and radiologic suites.

In other embodiments, at least a portion of the Isl1⁺/SNL⁺ atrial progenitors or their progeny as disclosed herein obtained from a subject can be stored for later implantation/infusion. The Isl1⁺/SNL⁺ atrial progenitors or their progeny as disclosed herein can be divided into more than one aliquot or unit such that part of the population of Isl1⁺/SNL⁺ atrial progenitors or their progeny are retained for later application while part is applied immediately to the subject. Moderate to long-term storage of all or part of the cells in a cell bank is also within the scope of this invention, as disclosed in U.S. Patent Application Serial No. 20030054331 and Patent Application No. WO03024215, and is incorporated by reference in their entireties. At the end of processing, the concentrated Isl1⁺/SNL⁺ atrial progenitors or their progeny as disclosed herein can be loaded into a delivery device, such as a syringe, for placement into the recipient by any means known to one of ordinary skill in the art.

### Pharmaceutical Compositions

The compositions as disclosed herein can further comprise an Isl1⁺/SNL⁺ atrial progenitor differentiation agent, for example a differentiation agent which promotes the differentiation of Isl1⁺/SNL⁺ atrial progenitor along cardiomyocyte lineages such as atrial myocyte and smooth muscle cells. Differentiation factors which promote the differentiation of cells into cardiomyocyte lineages are well known to those of ordinary skill in the art and are encompassed for use in the methods as disclosed herein. Examples of such agents include, but are not limited to, cardiotrophic agents, creatine, carnitine, taurine, cardiotropic factors as disclosed in U.S. Patent Application Serial No. 2003/0022367 which is incorporated herein by reference, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway. The pharmaceutical compositions may further comprise a pharmaceutically acceptable carrier.

The Isl1⁺/SNL⁺ atrial progenitors or their progeny as disclosed herein can be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. The Isl1⁺/SNL⁺ atrial progenitors or their progeny as disclosed herein can also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, the Isl1⁺/SNL⁺ atrial progenitors or their progeny as disclosed herein can be combined with a gene encoding pro-angiogenic and/or cardiomyogenic growth factor(s) which would allow cells to act as their own source of growth factor during cardiac repair or regeneration. Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid' adeno-associated virus. Isl1⁺/SNL⁺ atrial progenitors or their progeny could be implanted along with a carrier material bearing gene delivery vehicle capable of releasing and/or presenting genes to the implanted cells over time such that transduction can continue or be initiated. Particularly when the Isl1⁺/SNL⁺ atrial progenitors or their progeny are administered to a subject other than the subject from whom the Isl1⁺/SNL⁺ atrial progenitors or their progeny as disclosed herein were obtained, one or more immunosuppressive agents may be administered to the subject receiving the cells to prevent rejection of the transplanted Isl1⁺/SNL⁺ atrial progenitor cells. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 20020182211. In one embodiment, a immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the Isl1⁺/SNL⁺ atrial progenitors or their progeny as disclosed herein.

In certain embodiments of the invention, the Isl1⁺/SNL⁺ atrial progenitors or their progeny as disclosed herein are administered to a subject with one or more cellular differentiation agents, such as cytokines and growth factors, as disclosed herein. Examples of various cell differentiation agents are disclosed in U.S. Patent Application Serial No. 2003/0022367 which is incorporated herein by reference, or Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001. Other examples of cytokines and growth factors include, but are not limited to, cardiotrophic agents, creatine, carnitine, taurine, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway.

Pharmaceutical compositions comprising effective amounts of Isl1⁺/SNL⁺ atrial progenitors or their progeny as disclosed herein are also contemplated by the present invention. These compositions comprise an effective number of Isl1⁺/SNL⁺ atrial progenitors or their progeny, optionally, in combination with a pharmaceutically acceptable carrier, additive or excipient. In certain aspects of the present invention, cells are administered to the subject in need of a transplant in sterile saline. In other aspects of the present invention, the Isl1⁺/SNL⁺ atrial progenitors or their progeny are administered in Hanks Balanced Salt Solution (HBSS) or Isolyte S, pH 7.4. Other approaches may also be used, including the use of serum free cellular media. In one embodiment, the Isl1⁺/SNL⁺ atrial progenitors or their progeny are administered in plasma or fetal bovine serum, and DMSO. Systemic administration of the Isl1⁺/SNL⁺ atrial progenitors or their progeny to the subject can be preferred in certain indications, whereas direct administration at the site of or in proximity to the diseased and/or damaged tissue may be preferred in other indications.

The composition may optionally be packaged in a suitable container with written instructions for a desired purpose, such as the reconstitution of Isl1⁺/SNL⁺ atrial progenitors or their progeny to improve or correct a defect or disorder in cardiac function and/or of the cardiac muscle.

In one embodiment, the Isl1⁺/SNL⁺ atrial progenitors or their progeny as disclosed herein can be administered with a differentiation agent. In one embodiment, the Isl1⁺/SNL⁺ atrial progenitors or their progeny can be combined with the differentiation agent to administration into the subject. In another embodiment, the Isl1⁺/SNL⁺ atrial progenitors or their progeny can be administered separately to the subject from the differentiation agent. Optionally, if the Isl1⁺/SNL⁺ atrial progenitors or their progeny are administered separately from the differentiation agent, there is a temporal separation in the administration of the cells and the differentiation agent. The temporal separation may range from about less than a minute in time, to about hours or days in time. The determination of the optimal timing and order of administration is readily and routinely determined by one of ordinary skill in the art.

### Uses of Isl1⁺/SNL⁺ Atrial Progenitors as Assays

In one embodiment of the invention, the Isl1⁺/SNL⁺ atrial progenitors can be used as an assay for the study and understanding of signaling pathways of cardiomyocyte lineage differentiation. Also, the cardiomyocytes, such as atrial myocytes can be used in an assay to study and understanding of the signalling pathways of reprogramming to become Isl1⁺/SNL⁺ atrial progenitors. Furthermore, the Isl1⁺/SNL⁺ atrial progenitors and cardiomyocyte-derived Isl1⁺/SNL⁺ atrial progenitors can be used to aid the development of therapeutic applications for congenital and adult heart failure. The use of Isl1⁺/SNL⁺ atrial progenitors and cardiomyocyte-derived Isl1⁺/SNL⁺ atrial progenitors enable the study of specific cardiac lineages, in particular cardiac structures without the need and complexity of time consuming animal models. In another embodiment, the Isl1⁺/SNL⁺ atrial progenitors and cardiomyocyte-derived Isl1⁺/SNL⁺ atrial progenitors can be genetically modified to carry specific disease and/or pathological traits and phenotypes of cardiac disease and adult heart failure.

In one embodiment, the assay comprises a plurality of Isl1⁺/SNL⁺ atrial progenitors and cardiomyocyte-derived Isl1⁺/SNL⁺ atrial progenitors, or their progeny. In one embodiment, the assay comprises Isl1⁺/SNL⁺ atrial progenitors derived from the cardiomyocytes. In one embodiment, the assay can be used for the study of differentiation pathways of Isl1⁺/SNL⁺ atrial progenitors, for example but not limited to the differentiation along the cardiomyocyte lineages, smooth muscle lineages and subpopulations of these lineages. In one embodiment, the study of subpopulations can be, for example, study of subpopulations of cardiomyocytes, for example artial cardiomyocytes, ventricular cardiomyocytes, outflow tract cardiomyocytes, conduction system cardiomyocytes.

In another embodiment, the assay can be used to study Isl1⁺/SNL⁺ atrial progenitors as disclosed herein which comprise a pathological characteristic, for example, a disease and/or genetic characteristic associated with a disease or disorder. In some embodiments, the disease of disorder is a cardiovascular disorder or disease. In some embodiments, the cardiovascular stem cell has been genetically engineered to comprise the characteristic associated with a disease or disorder. Such methods to genetically engineer Isl1⁺/SNL⁺ atrial progenitors are well known by those in the art, and include introducing nucleic acids into the cell by means of transfection, for example but not limited to use of viral vectors or by other means known in the art.

In some embodiments, the Isl1⁺/SNL⁺ atrial progenitors can be easily manipulated in experimental systems that offer the advantages of targeted lineage differentiation as well as clonal homogeneity and the ability to manipulate external environments. Furthermore, due to ethical unacceptability of experimentally altering a human germ line, the ES cell transgenic route is not available for experiments that involve the manipulation of human genes. Gene targeting in human Isl1⁺/SNL⁺ atrial progenitors as disclosed herein allows important applications in areas where rodent model systems do not adequately recapitulate human biology or disease processes.

In another embodiment, the Isl1⁺/SNL⁺ atrial progenitors can be used to prepare a cDNA library relatively uncontaminated with cDNA that is preferentially expressed in cells from other lineages. For example, Isl1⁺/SNL⁺ atrial progenitors can be generated, for example from reprogramming cardiomyocytes to become Isl1⁺/SNL⁺ atrial progenitors, and such Isl1⁺/SNL⁺ atrial progenitors are collected and then mRNA is prepared from the pellet by standard techniques (Sambrook et al., supra). After reverse transcribing into cDNA, the preparation can be subtracted with cDNA from other undifferentiated ES cells, other progenitor cells, or end-stage cells from the cardiomyocyte or any other developmental pathway, for example, in a subtraction cDNA library procedure.

The Isl1⁺/SNL⁺ atrial progenitors can also be used to prepare antibodies that are specific for markers of Isl1⁺/SNL⁺ atrial progenitors. Polyclonal antibodies can be prepared by injecting a vertebrate animal with cells of this invention in an immunogenic form. Production of monoclonal antibodies is described in such standard references as U.S. Pat. Nos. 4,491,632, 4,472,500 and 4,444,887, and Methods in Enzymology 73B:3 (1981). Specific antibody molecules can also be produced by contacting a library of immunocompetent cells or viral particles with the target antigen, and growing out positively selected clones. See Marks et al., New Eng. J. Med. 335:730, 1996, and McGuiness et al., Nature Biotechnol. 14:1449, 1996. A further alternative is reassembly of random DNA fragments into antibody encoding regions, as described in EP patent application 1,094,108 A.

The antibodies in turn can be used to identify or rescue (for example restore the phenotype) Isl1⁺/SNL⁺ atrial progenitors from a mixed cell population, for purposes such as co-staining during immunodiagnosis using tissue samples, and identifying Isl1⁺/SNL⁺ atrial progenitors from the reprogramming of terminally differentiated cardiomyocytes. Of particular interest is the examination of the gene expression profile during and following reprogramming of cardiomyocytes to Isl1⁺/SNL⁺ atrial progenitors. The expressed set of genes may be compared against other subsets of progenitor cells, against ES cells, against adult heart tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+mRNA. One of skill in the art can readily use these methods to determine differences in the molecular size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680. Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505. Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined. Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes. Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992. General methods in molecular and cellular biochemistry can also be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following written description provides exemplary methodology and guidance for carrying out many of the varying aspects of the present invention.

Molecular Biology Techniques: Standard molecular biology techniques known in the art and not specifically described are generally followed as in Sambrook et al., Molecular Cloning: A Laboratory Manual, Cold Springs Harbor Laboratory, N.Y. (1989, 1992), and in Ausubel et al., Current Protocols in Molecular Biology, John Wiley and Sons, Baltimore, Md. (1989). Polymerase chain reaction (PCR) is carried out generally as in PCR Protocols: A Guide to Methods and Applications, Academic Press, San Diego, Calif. (1990). Reactions and manipulations involving other nucleic acid techniques, unless stated otherwise, are performed as generally described in Sambrook et al., Molecular Cloning: A Laboratory Manual, Cold Springs Harbor Laboratory Press, and methodology as set forth in U.S. Pat. Nos. 4,666,828; 4,683,202; 4,801,531; 5,192,659; and 5,272,057 and incorporated herein by reference. In situ PCR in combination with Flow Cytometry can be used for detection of cells containing specific DNA and mRNA sequences (see, for example, Testoni et al., Blood, 1996, 87:3822).

Immunoassays: Standard methods in immunology known in the art and not specifically described are generally followed as in Stites et al. (Eds.), Basic And Clinical Immunology, 8th Ed., Appleton & Lange, Norwalk, Conn. (1994); and Mishell and Shigi (Eds.), Selected Methods in Cellular Immunology, W. H. Freeman and Co., New York (1980).

In general, immunoassays are employed to assess a specimen such as for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIA), can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al., Molecular Cloning: A Laboratory Manual, Cold Springs Harbor, N.Y., 1989. Numerous other references also may be relied on for these teachings.

Further elaboration of various methods that can be utilized for quantifying the presence of the desired marker include measuring the amount of a molecule that is present. A convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein (GFP) chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

Antibody Production: Antibodies may be monoclonal, polyclonal, or recombinant. Conveniently, the antibodies may be prepared against the immunogen or immunogenic portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Springs Harbor, N.Y. (1988) and Borrebaeck, Antibody Engineering—A Practical Guide by W. H. Freeman and Co. (1992). Antibody fragments may also be prepared from the antibodies and include Fab and F(ab′)2 by methods known to those skilled in the art. For producing polyclonal antibodies a host, such as a rabbit or goat, is immunized with the immunogen or immunogenic fragment, generally with an adjuvant and, if necessary, coupled to a carrier; antibodies to the immunogen are collected from the serum. Further, the polyclonal antibody can be absorbed such that it is monospecific. That is, the serum can be exposed to related immunogens so that cross-reactive antibodies are removed from the serum rendering it monospecific.

For producing monoclonal antibodies, an appropriate donor is hyperimmunized with the immunogen, generally a mouse, and splenic antibody-producing cells are isolated. These cells are fused to immortal cells, such as myeloma cells, to provide a fused cell hybrid that is immortal and secretes the required antibody. The cells are then cultured, and the monoclonal antibodies harvested from the culture media.

For producing recombinant antibodies, messenger RNA from antibody-producing B-lymphocytes of animals or hybridoma is reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system. Antibody cDNA can also be obtained by screening pertinent expression libraries. The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982). The binding of antibodies to a solid support substrate is also well known in the art. (see for a general discussion Harlow & Lane, Antibodies: A Laboratory Manual, Cold Spring Harbor Laboratory Publications, New York, 1988 and Borrebaeck, Antibody Engineering—A Practical Guide, W. H. Freeman and Co., 1992). The detectable moieties contemplated with the present invention can include, but are not limited to, fluorescent, metallic, enzymatic and radioactive markers. Examples include biotin, gold, ferritin, alkaline phosphates, galactosidase, peroxidase, urease, fluorescein, rhodamine, tritium, 14C, iodination and green fluorescent protein.

Gene therapy and genetic engineering of cardiovascular stem cells and/or mesenchymal cells: Gene therapy as used herein refers to the transfer of genetic material (e.g., DNA or RNA) of interest into a host to treat or prevent a genetic or acquired disease or condition. The genetic material of interest encodes a product (e.g., a protein, polypeptide, and peptide, functional RNA, antisense, RNA, microRNA, siRNA, shRNA, PNA, pcPNA) whose in vivo production is desired. For example, the genetic material of interest encodes a hormone, receptor, enzyme polypeptide or peptide of therapeutic value. Alternatively, the genetic material of interest encodes a suicide gene. For a review see “Gene Therapy” in Advances in Pharmacology, Academic Press, San Diego, Calif., 1997.

With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Feral. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & Isenberg eds, CRC Press 1989), Heart Development (Harvey & Rosenthal, Academic Press 1998).

In some embodiments of the present invention may be defined in any of the following numbered paragraphs:

1. A method for isolating atrial progenitors, the method comprising contacting a population of progenitor cells with agents reactive to Islet 1 and SLN, and separating reactive positive cells from non-reactive cells.
2. A method for isolating atrial progenitors, the method comprising introducing a reporter gene operatively linked to the regulatory sequence for Islet1 and SLN and separating the reactive positive cells expressing the reporter gene from non-reactive cells.
3. The method of paragraph 1 or 2, wherein the atrial progenitors are capable of differentiating into cells with a muscle cell or cardiomyocyte phenotypes.
4. The method of paragraph 3, wherein the cardiomyocyte phenotypes is an atrial myocyte.
5. The method of paragraph 4, wherein the atrial myocyte is a cTnT-positive, SLN-positive, Islet1-negative and MLC2v-negative atrial myocyte.
6. The method of paragraph 3, wherein the muscle cell phenotype is a smooth muscle cell.
7. The method of paragraph 6, wherein the smooth muscle cell is a smMHC-positive, Islet1-negative, cTnT-negative and SLN-negative smooth muscle cell.
8. The method of paragraph 1, wherein the agent is reactive to a nucleic acid encoding Islet 1 or SLN.
9. The method of paragraph 8, wherein the nucleic acid is selected from the group consisting of: RNA, messenger RNA (mRNA) and genomic RNA.
10. The method of paragraph 1, wherein the agent is a nucleic acid agent or a protein or fragment thereof.
11. The method of paragraph 10, wherein the nucleic acid is selected from a group consisting of DNA, RNA, PNA or pcDNA.
12. The method of paragraph 1, wherein the agent is reactive to the expression products of nucleic acids encoding Islet1 or SLN.
13. The method of paragraph 12, wherein the agent is a nucleic acid agent or protein or fragment thereof.
14. The method of paragraph 13, wherein the protein is an antibody or antibody fragment.
15. The method of paragraph 1, wherein the agent is a small molecule or aptamer.
16. The method of paragraph 2, wherein the reporter gene encodes fluorescence activity and/or chromogenic activity.
17. The method of paragraph 16, wherein the reporter gene encodes a fluorescent protein or fragment thereof.
18. The method of paragraph 17; wherein the fluorescent protein is detected by fluorescence cell sorting (FACS), fluorimetry, and/or microscope techniques.
19. The method of paragraph 2, wherein in the separating is done by fluorescence cell sorting (FAC).
20. The method of paragraph 19, wherein the reporter gene encodes an enzyme.
21. The method of paragraph 20; wherein the enzyme is selected from a group consisting of; beta-galactosidase (β-gal); beta-lactamase; dihydrofolate reductase (DHFR); luciferase; chloroamphenicol acetyl transferase, beta-glucosidase, beta-glucuronidase and modifications and fragments thereof.
22. The method of paragraph 2, wherein in the regulatory sequence is a promoter sequence or part of a promoter sequence thereof sufficient to direct transcription.
23. The method of paragraph 22, wherein the reporter gene is a resistance gene.
24. A method to generate a Isl1+/SLN+ atrial progenitor cell, the method comprising culturing least one atrial myocyte cell in the presence of a cardiac messenchymal cell feeder layer for a sufficient period of time for an atrial myocyte cell to retrodifferentiate into Isl1+/SLN+ atrial progenitor cell.
25. The method of paragraph 24, wherein the atrial myocyte is a mature atrial myocyte cell.
26. The method of paragraph 25, wherein the mature atrial myocyte cell is a cTnT-positive, SLN-positive, Islet1-negative and MLC2v-negative mature atrial myocyte.
27. The method of paragraph 22, wherein the atrial myocyte cell is from a mammal.
28. The method of paragraph 27, wherein the mammal is a human.
29. The method of paragraph 27, wherein the mammal is a transgenic animal or a genetically modified animal.
30. The method of paragraph 24, wherein the atrial myocyte cell is a genetically modified atrial myocyte cell.
31. The method of paragraph 30, wherein the genetically modified atrial myocyte cell comprises a gene to provide the atrial myocyte with a desired phenotype.
32. The method of paragraphs 30 or 31, wherein the genetically modified atrial myocyte cell comprises a reporter gene for phenotypic identification.
33. The method of paragraphs 32, wherein the reporter gene is operatively linked to a promoter.
34. The method of paragraph 33, wherein the promoter is an inducible promoter.
35. The method of paragraph 33, wherein the promoter is a tissue specific promoter.
36. The method of paragraph 35, wherein the tissue specific promoter an Isl1 promoter and/or SLN promoter or fragment thereof.
37. The method of paragraphs 30 or 31, wherein the genetically modified atrial myocyte cell comprises a therapeutic nucleic acid sequence.
38. A method to generate a Isl1+/SLN+atrial progenitor cell, the method comprising culturing least one Isl1+ progenitor cell in the presence of a cardiac messenchymal cell feeder layer for a sufficient period of time for a Isl1+ cell to differentiate into Isl1+/SLN+atrial progenitor cell.
39. The method of paragraph 38, wherein the immature cardiac progenitor cell is an immature cardiac progenitor cell.
40. The method of paragraph 39, wherein the immature cardiac progenitor cell is a Isl1-positive (Isl1+), SLN-positive (SLN+) immature cardiac progenitor cell.
41. The method of paragraphs 38 to 40, wherein the immature cardiac progenitor cell is from a mammal.
42. The method of paragraph 41, wherein the mammal is a human.
43. The method of paragraph 42, wherein the mammal is a transgenic animal or a genetically modified animal.
44. The method of paragraph 38, wherein the Isl1+ progenitor cell is a genetically modified Isl1+ progenitor cell.
45. The method of paragraph 44, wherein the genetically modified Isl1+ progenitor cell comprises a gene to provide the Isl1+ progenitor cell with a desired phenotype.
46. The method of paragraphs 44 or 45, wherein the genetically modified Isl1+ progenitor cell comprises a reporter gene for phenotypic identification.
47. The method of paragraph 46, wherein the reporter gene is operatively linked to a promoter.
48. The method of paragraph 47, wherein the promoter is an inducible promoter.
49. The method of paragraph 47, wherein the promoter is a tissue specific promoter.
50. The method of paragraph 49, wherein the tissue specific promoter an Isl1 promoter and/or SLN promoter or fragment thereof.
51. The method of paragraphs 44 or 45, wherein the genetically modified Isl1+ progenitor cell comprises a therapeutic nucleic acid sequence.
52. A composition comprising an isolated population of Islet1+, SLN+ atrial progenitor cells.
53. The composition of paragraph 37, wherein the Islet1+, SLN+ atrial progenitor cells are generated according to the methods of paragraphs 24 to 37 and/or 38 to 51.
54. The composition of paragraph 37, wherein the Islet1+, SLN+ atrial progenitor cells are identified according to the methods of paragraphs 1 to 23.
55. A clonal cell line produced by the methods set forth in any of the paragraphs 24 to 37 and/or 38 to 51.
56. The clonal cell line of paragraph 55, wherein the cells are subsequently cryopreserved.
57. A method to generate a population of smooth muscle cells and/or cardiomyocytes cells, the method comprising culturing at least one Isl1+/SLN+atrial progenitor in the presence of a cardiac messenchymal cell feeder layer for a sufficient period of time for the Isl1+/SLN+ atrial progenitor to proliferate and differentiate into smooth muscle cells and/or cardiomyocytes cells, wherein a population of smooth muscle cells and/or cardiomyocytes cells is generated.
58. The method of paragraph 57, wherein the Isl1+/SLN+ atrial progenitor are generated by any of the paragraphs 24 to 37 and/or 38 to 51.
59. The method of paragraph 57, wherein the Isl1+/SLN+ atrial progenitor are identified by any of the paragraphs 1 to 23.
60. The method of paragraph 57, wherein the Isl1+/SLN+ atrial progenitors are cultured at a clonal density on the cardiac mesenchymal feeder layer.
61. The method of paragraph 57, wherein the cardiomyocyte is an atrial myocyte.
62. The method of paragraph 61, wherein the atrial myocyte is a cTnT-positive (cTnT+), SLN-positive (SLN+), Islet1-negative (Isl1−) and MLC2v-negative (MLC2v−) atrial myocyte.
63. The method of paragraph 57, wherein the smooth muscle cell is a smMHC-positive (smMHC+), Islet1-negative (Isl1−), cTnT-negative (cTnT−) and SLN-negative (SLN−) smooth muscle cell.
64. A method for enhancing cardiac function in a subject, the method comprising administering to the subject a composition comprising Isl1+/SLN+ atrial progenitors generated by the methods as set forth in any of the proceeding paragraphs, wherein the composition comprising Isl1+/SLN+ atrial progenitors enhances cardiac function in a subject.
65. A method of paragraph 64, wherein the subject suffers from a disease or disorder characterized by insufficient cardiac function.
66. The methods of paragraph 64, further defined as;
(i) obtaining an atrial myocyte from the subject;
(ii) generating a Isl1+/SLN+ atrial progenitor by the methods as set forth in paragraphs 24 to 37 and/or 38 to 51; and
(iii) transplanting a population of Isl1+/SLN+ atrial progenitors from step (ii) or their progeny into a subject in an effective amount to treat a disorder characterized by insufficient cardiac function.
67. The methods of paragraph 66, wherein the Isl1+/SLN+ atrial progenitors from step (ii) can be optionally genetically manipulated prior to step (iii) to comprise a gene to provide a Isl1+/SLN+ atrial progenitors with a desired phenotype.
68. The method of paragraph 67, wherein the genetically modified Isl1+/SLN+ atrial progenitor comprises a reporter gene for phenotypic identification.
69. The method of paragraph 68, wherein the reporter gene is operatively linked to a promoter.
70. The method of paragraph 69, wherein the promoter is an inducible promoter.
71. The method of paragraph 69, wherein the promoter is a tissue specific promoter.
72. The method of paragraph 71, wherein the tissue specific promoter an Isl1 promoter and/or SLN promoter or a fragment thereof.
73. The method of paragraph 67, wherein the genetically modified Isl1+/SLN+ atrial progenitor comprises a therapeutic nucleic acid sequence.
74. The method of paragraph 73, wherein the therapeutic nucleic acid sequence encodes at least one therapeutic protein or polypeptide and/or at least one inhibitory nucleic acid sequence.
75. The method of paragraph 74, wherein the inhibitory nucleic acid is selected from the group consisting of: RNA, DNA, PNA, pcPNA; siRNA; miRNA, shRNA., locked nucleic acid (LNA).
76. The method of paragraph 65, wherein the disease or disorder is congestive heart failure, myocardial infarction, tissue ischemia, cardiac ischemia, vascular disease, acquired heart disease, congenital heart disease, atherosclerosis, cardiomyopathy, dysfunctional conduction systems, dysfunctional coronary arteries, pulmonary heard hypertension,
77. The method of paragraph 65, wherein the disease is selected from the group consisting of congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease.
78. The method of paragraph 65, wherein the subject is a mammal.
79. The method of paragraph 78, wherein the mammal is a human.
80. The method of paragraph 65, wherein the subject has suffered myocardial infarction.
81. The method of paragraph 65, wherein the subject has or is at risk of heart failure.
82. The method of paragraph 81, wherein the heart failure is acquired heart failure.
83. The method of paragraph 81, wherein the heart failure is associated with atherosclerosis, cardiomyopathy, congestive heart failure, myocardial infarction, ischemic diseases of the heart, atrial and ventricular arrhythmias, hypertensive vascular diseases, peripheral vascular diseases.
84. The method of paragraph 65, wherein the subject has a congenital heart disease.
85. The method of paragraph 84, wherein the subject has a condition selected from a group consisting of: hypertension; blood flow disorders; symptomatic arrhythmia; pulmonary hypertension; arthrosclerosis; dysfunction in conduction system; dysfunction in coronary arteries; dysfunction in coronary arterial tree and coronary artery colaterization.
86. The method of paragraph 65, wherein enhancing cardiac function is a method to treat or prevent heart failure.
87. The method of paragraph 65, wherein the composition is administered via endomyocardial, epimyocardial, intraventricular, intracoronary, retrosinus, intra-arterial, intra-pericardial, or intravenous administration route.
88. The method of paragraph 65, wherein the composition is administered to the subject's vasculature.
89. The method of paragraph 65, wherein the cells are harvested from the same subject to which the composition is administered.
90. The method of paragraph 65, wherein the Isl1+/SNL+ atrial progenitor is genetically modified such that the expression of at least one gene is altered in the Isl1+/SNL+ atrial progenitor before being administered to the subject.
91. A cell of Isl1+/SNL+ atrial progenitor lineage generated by the methods set forth in any of paragraphs 24 to 37 and/or 38 to 51 for the treatment or prevention of a cardiovascular disease or disorder in a subject.
92. A smooth muscle cell or cardiomyocyte cell generated by the methods set forth in any of paragraphs 57-63 2 for the treatment or prevention of a cardiovascular disease or disorder in a subject.
93. The cells of paragraphs 91 or 92, wherein the subject is a mammal.
94. The cells of paragraphs 91 or 92, wherein the subject is a human.
95. The cells of paragraphs 91 or 92, wherein the cell is a mammalian cell.
96. The cells of paragraphs 91 or 92, wherein the subject has suffered myocardial infarction.
97. The cells of paragraphs 91 or 92, wherein the subject has or is at risk of heart failure.
98. The cells of paragraph 97, wherein the heart failure is acquired heart failure.
99. The cells of paragraphs 98, wherein the heart failure is associated with atherosclerosis, cardiomyopathy, congestive heart failure, myocardial infarction, ischemic diseases of the heart, artrial and ventricular arrhythmias, hypertensive vascular diseases, peripheral vascular diseases.
100. The cells of paragraphs 91 or 92, wherein the subject has a congenital heart disease.
101. The cells of paragraphs 91 or 92, wherein the subject has a condition selected from a group consisting of: hypertension; blood flow disorders; symptomatic arrhythmia; pulmonary hypertension; arthrosclerosis; dysfunction in conduction system; dysfunction in coronary arteries; dysfunction in coronary arterial tree and coronary artery colaterization.
102. A method for identifying agents which promote the retrodifferention of a cardiomyocyte cell to a Isl1+/SLN+ atrial progenitor, the method comprising;
(i) culturing a population of cardiomyocyte cells and contacting at least one cardiomyocyte cell with one or more agents; and
(ii) monitoring the expression of Isl1+ and SLN+ in the cardiomyocyte cell;
wherein an agent which results the expression of both Isl1+ and SLN+ in the cardiomyocyte cell identifies an agent which promotes the retrodifferention of a cardiomyocyte cell to a Isl1+/SLN+ atrial progenitor cell.
103. The method of paragraph 102, wherein the agent is a nucleic acid or a nucleic acid analogue.
104. The method of paragraph 103, wherein the nucleic acid encodes a polypeptide.
105. The method of paragraph 102, wherein the nucleic acid is selected from the group consisting of; RNA, DNA, PNA, pcPNA, RNAi, siRNA, miRNA, shRNA, stRNA, locked nucleic acid (LNA).
106. The method of paragraph 102, wherein the monitoring for the expression of Isl1+ and SLN+ is identification of a Isl1+/SLN+ atrial progenitor cell according to the methods of any of paragraphs 1 to 23.

The present invention is further illustrated by the following examples which in no way should be construed as being further limiting, The contents of all cited references, including literature references, issued patents, published patent applications, and co-pending patent applications, cited throughout this application are hereby expressly incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

The examples presented herein relate to the methods and compositions for the identification of Isl1+/SLN+ atrial progenitors, and a method to generate Isl1+/SLN+ atrial progenitors from atrial myocytes or immature Isl1+ progenitor cells. The examples also relate to the differentiation of Isl1+/SNL+ cells into cardiomyocyte cells, such as atrial myocytes as well as smooth muscle cells, and the prevention and/or treatment of cardiovascular disorders and diseases. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Methods

Generation of SLN-Cre mice. Exon 2 of SLN locus including 1^{st} ATG was replaced with Cre cDNA. A correctly targeted R1 ES clone was used to generate chimeric mice.

Preparation of primary myocytes and cardiac mesenchymal feeder layer. Neonatal hearts were predigested with 0.5 mg/ml trypsin in HBSS at 4 C overnight followed by strong digestion with collagenase at 37 C for 1 hour (0.5 mg/ml in HBSS). Cardiac mesenchymal fibroblasts were separated from myocytes by differential plating for 1 hour twice. Fibroblasts from first and second differential plating were combined, grown until confluent and treated with 10 μm/ml mitomycin C for 2 hours on the day before progenitors were seeded. The contamination of myocytes in the fibroblast fraction was less than 0.07% by cTnT staining.

Atrial ablation models. Open-chest atrial injury was performed according to the protocol approved by IACUC. Briefly, the 3-4 week-old female rats were anesthetized using Xylazine 5-10 mg/kg and Ketamine 80-100 mg/kg/bw ip. The animals were then positioned on an operating table for the intubation and mechanical ventirlation. The chest cavity was opened under the intubation and mechanical ventilation. After exposing the heart, left atria were injured by ligation with nylon sutures. The sham operation mice underwent the same procedure without ligation. Genetic ablation models were obtained by MLP^{-/-} mice¹⁹, or by αMHC^{mCm/+}; Ryr2^{flox/flox} mice injected with 75 mg/kg TAM²⁰.

Histology and immunostaining. Whole mount and section Xgal stainings were performed according to standard protocols. Double staining for Xgal and immunostaining were performed as follows: 8 um frozen section or cells were stained with Xgal followed by postfixation for 5 min, 0.3% hydrogen peroxide treatment for 15 min, blocking with 10% normal goat serum for 1 hour and antibody reaction in 3% normal goat serum at 4 C overnight. Secondary antibody reaction was performed with Vectastain ABC kit (Vector lab) according to the manufacturers protocol. Section Xgal/Isl1 staining was performed as previously described²¹. The concentrations of the primary antibodies are; cTnT (1:200, Lab Vision Corp., Fremont, Calif.), smMHC (1:500, Biomedical Technologies Inc., Stoughton, Mass.), αSMA (1:500, DAKO, Carpinteria, Calif.), Isl1 (1:200, DSHB, Iowa City, Iowa), DsRed (1:500, Clontech, Mountain View, Calif.), BrdU (1:1000, Abcam, Cambridge, Mass.) and MLC2v (1:200, Axxora, San Diego, Calif.).

Electron microscopic analysis. Tissues were fixed in 2.0% glutaraldehyde in 0.1 M sodium cacodylate buffer, pH 7.4 (Electron Microscopy Sciences, Hatfield, Pa.) overnight at 4 C. They were rinsed in buffer, post-fixed in 1% osmium tetroxide in cacodylate buffer for one hour at room temperature, rinsed in buffer again, then in distilled water and stained, en bloc, in an aqueous solution of 2.0% uranyl acetate for one hour at room temperature. They were rinsed in distilled water and dehydrated through a graded series of ethanol to 100%. They were then infiltrated with Epon resin (Ted Pella, Redding, Calif.) in a 1:1 solution of Epon:ethanol. The following day they were placed in fresh Epon for several hours and then embedded in Epon overnight at 60° C. Thin sections were cut on a Reichert Ultracut E ultramicrotome, collected on formvar-coated grids, stained with uranyl acetate and lead citrate and examined in a JEOL JEM 1011 transmission electron microscope at 80 kV. Images were collected using an AMT (Advanced Microscopy Techniques, Danvers, Mass.) digital imaging system.

Isolation and culture of embryonic atrial progenitors. Atrial tissues from pregnant R26R heterozygotes crossed with SLN^{Cre/+} male were dissected and dissociated with 10 mM collagenase B and 10 mM collagenase D (Roche Diagnostics, Indianapolis, Ind.) in HEPES-buffered saline with 20% FCS at 37 C for 1 hour. Single cell suspension was plated onto cardiac mesenchymal feeder at a clonal density (5K cells/ml) in ES medium (15% FCS, 2500 i.u./ml penicillin/streptomycin, 200 mM L-glutamine, Non-essential amino acid, 2-ME). For cardiac and smooth muscle differentiation, cells were cultured in dark media (10% horse serum, 5% FCS, 5 mM HEPES, 5000 i.u./ml penicillin/streptomycin, 200 mM L-glutamine) with or without B27 (Invitrogen, Carlsbad, Calif.).

Isolation and culture of neonatal atrial myocytes. Neonatal atria were dissected from neonates born from R26R female crossed with SLN^{Cre/+} or Isl1^{mCm/+} male, and dissociated as described above. After second differential plating, floating cardiomyocytes were collected and seeded onto cardiac mesenchymal feeder in ES medium for expansion or fibronectin-coated plate in dark medium with or without B27 for differentiation. Contamination of ventricular myocytes was 0.10% by MLC2v staining on the next day. Contamination of non-myocytes is about 10%. 4OH-TAM (Sigma, St. Louis, Mo.) was used at the concentration of 0.2 mM when Isl1^{mCm/+} male were used. Adult atrial cardiomyocytes were isolated by enzymatic digestion as previously described²².

PCR and qPCR. RNA was extracted with Trizol (Invitrogen, Carlsbad, Calif.) or Absolute nanoprep kit (Stratagene, Ceder Creek, Tex.) according to manufacturer's protocol, and cDNAs were synthesized with iScript kit (BioRad, Hercules, Calif.). Colony PCR was run for 35 cycles. Quantitative PCR was performed with SYBR Green system and i-Cycler (BioRad, Hercules, Calif.). Primer sequences are available upon request to A.N. or H.N.

Ca transient assay. Isolated cardiomyocytes were loaded with Fura-2, perfused with Tyrode buffer, and [Ca²⁺]i transients were recorded as changes in Fura-2 ratio (340/380 nm) using a spectrofluoroscope system (Ionoptix, Milton Mass.).

### Example 1

In the heart, SLN is a regulator of the sarco(endo)plasmic reticulum Ca²⁺ ATPase that is specifically expressed in atrial muscle.

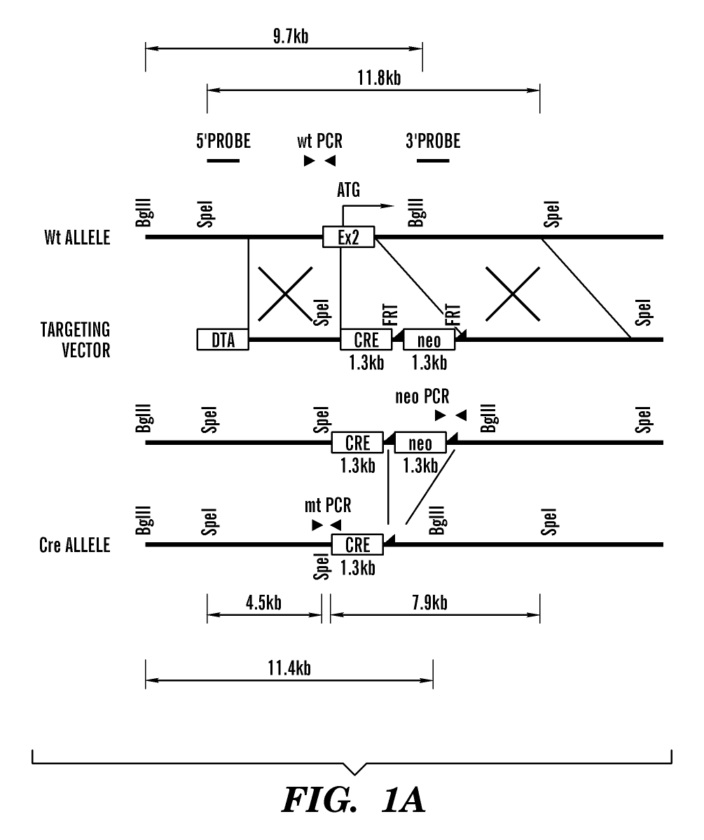

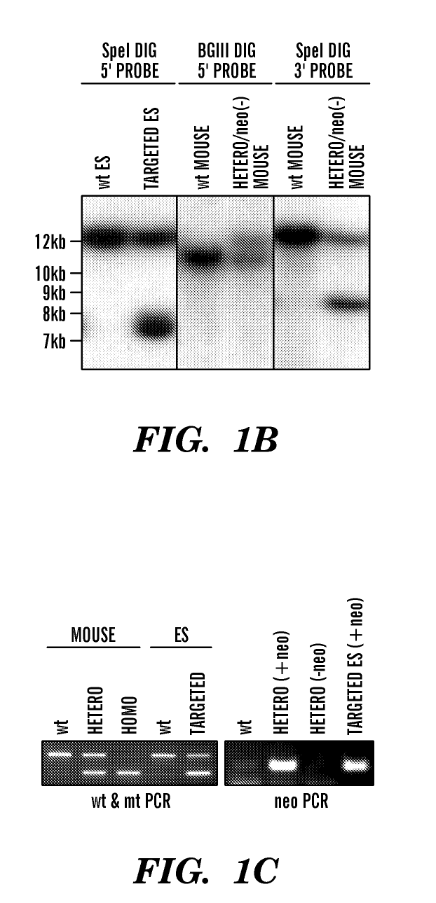

To generate an atrial-specific deleter line, the inventors introduced Cre recombinase by homologous recombination into exon 2 of the SLN locus (FIGS. 1A, 1B, 1C). In the heart, SLN is a regulator of the sarco(endo)plasmic reticulum Ca²⁺-ATPase that is specifically expressed in atrial muscle^{8, 9}. SLN^{cre/+} heterozygotes displayed no morphological or fertility defects. To trace the cell fate of embryonic atrial lineages, the inventors analyzed SLN^{cre/+}; R26R embryos and postnatal hearts. While SLN mRNA is expressed at E10.0, the βgal activity was first detected in the atria and the dermamyotome at around E10.5, when Isl1 is still positive in atrial lineage (data not shown)¹⁰. Using In situ hybridization for SLN at E10.0 combined with immunohistochemistry with X-gal staining, the inventors demonstrated that SLN lineage contributed to atria. Specifically, the inventor determined atrial specific labeling by In situ hybridization for SLN (E10.0), whole mount Xgal staining (E10.5, E12.5, neonatal heart and adult heart), section Xgal staining (neonatal heart) and whole mount fluorescence of adult heart and discovered that βgal and DsRed expression was restricted to atrial myocytes throughout cardiogenesis and in the adult heart. In the postnatal heart, the atrial myocardium was broadly and strongly labeled (data not shown). No Xgal-positive cells were found in the endocardium or epicardium. Double staining for Xgal and markers for the conduction system on serial sections indicated that the SA nodal cells mainly originate from the atrial lineage in contrast to AV nodal cells that originate from ventricular lineage¹¹ (data not shown).

Xgal analysis in the inflow region visualized the anatomical distribution of the myocardial sleeves of the pulmonary veins (PVs) and venae cavae was performed (data not shown). The Xgal staining extended up to bifurcation of internal carotid and subclavian veins in the cranial region and down to the diaphragm in the thoracic cavity. The boundary of the right atrium and the venae cavae is demarcated by venous valves that also are derived from SLN-expressing cells. Using immunohistochemistry for Xgal with immunostaining for HCN4 and AChE on adjacent serial sections of SLNcre/+; R26R neonates, the inventors demonstrated SLN lineage contributed to conduction system and to cardiac inflow. Staining of Xgal, HCN4 and AChE on adjacent serial sections of SLNcre/+; R26R neonate demonstrated that most of the SA nodal cells but not AV nodal cells are derived from SLN-expressing cells (data not shown). The inventors then also demonstrated SLN lineage contributes to cardiac inflow using whole mount Xgal staining of the inflow tract of SLNcre/+; R26R embryo at E13.5 and adult heart, and discovered that the proximal part of the SVC, IVC and PV are derived from atrial lineages. The distal ends of the inlets taper off toward the periphery, forming myocardial sleeves. Myocardial sleeves extend up to the bifurcation of jugular vein and subclavian vein and down to the diaphragm level. The proximal domain of the vena cava (VC) consists of two muscular layers, myocardial and smooth muscle layers, and demarcated from right atrium (RA) by venous valves (VV). The inventors thus discovered that whereas the muscular layer of the atrial chamber proximal to the venous valves consist only of myocardial cells, the vascular walls of the proximal superior and inferior venae cavae (SVC and IVC) distal to the venous valves consist of two muscular layers—the outer myocardial layer derived from SLN-expressing cells and inner smooth muscle layer positive for smMHC, a definitive marker for vascular smooth muscle cells (data not shown). The myocardial layer tapers off towards the periphery and generates myocardial sleeves in the great veins. The boundary between the pulmonary vein and the left atrium is not anatomically discrete, but Xgal/smMHC analyses revealed a clear border between them. Similar to the venae cavae, the proximal part of PV was composed of a two layer structure. Thus, SLN-cre knock-in mouse line is a reliable model for tracking atrial lineage and analyzing the tissue structure of inflow region precisely (Table 1).

TABLE 1
shows a summary of lineage
contribution of atrial progenitors.

| Working Myocyte | |
|---|---|
| Atrial myocytes | 95 − 100% |
| Ventricular myocytes | — |
| Conduction System | |
| Sinoatrial node | — |
| Atrioventricular node | 95 − 100% |
| Purkinje fiber | — |
| Endocardium | — |
| Epicardium | — |
| Cardiac ganglia | — |
| Blood vessels | |
| Endothelium | — |
| Smooth muscle | |
| Aorta | — |
| Pulmonary trunk | — |
| Large Coronary | — |
| Superior vena cava | 5 − 10% |
| Inferior vena cava | 5 − 10% |
| Pulmonary vein | 5 − 10% |
| Valves | |
| Mitral valve | — |
| Tricuspid valve | — |
| Aortic valve | — |
| Pulmonary valve | — |
| Venous Valves between | 80 − 90% |
| RA and vena cava | |

Further analysis revealed the close relationship between cardiac and smooth muscle lineages during cardiovascular development. Double staining and serial section analysis demonstrated that Xgal-positive cells were also found in the smMHC-positive smooth muscle layer of the inflow region. This discovery was confirmed by confocal analysis using SLN^{cre/+}; CAG-DsRed reporter mice double-stained with anti-DsRed and anti-smMHC antibodies. In particular, the inventors demonstrated smooth muscle was contributed by atrial progenitors by discovering that Xgal and smMHC are expressed in the right atrium of the heart from adult SLN^{cre/+}; R26R mouse, by demonstrating that some of the smMHC-positive smooth muscle cells in the inner layer are costained with Xgal. Electron microscopic analysis of serial sections indentified that Xgal deposits in smooth muscle cells (SMC) with non-striated myofilaments (MF). Additionally, confocal microscopic analysis of PV of SLN^{cre/+}×CAG-DsRed reporter adult mice double immunostaining of DsRed and smMHC identified peripheral PV lacking the myocardial sheath and the expression of a smooth muscle marker in βgal-labeled cells isolated from cardiac inflow region of adult cre/+; R26R mice.

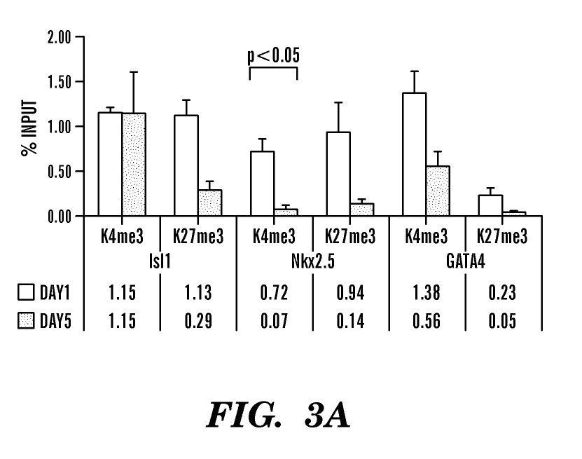

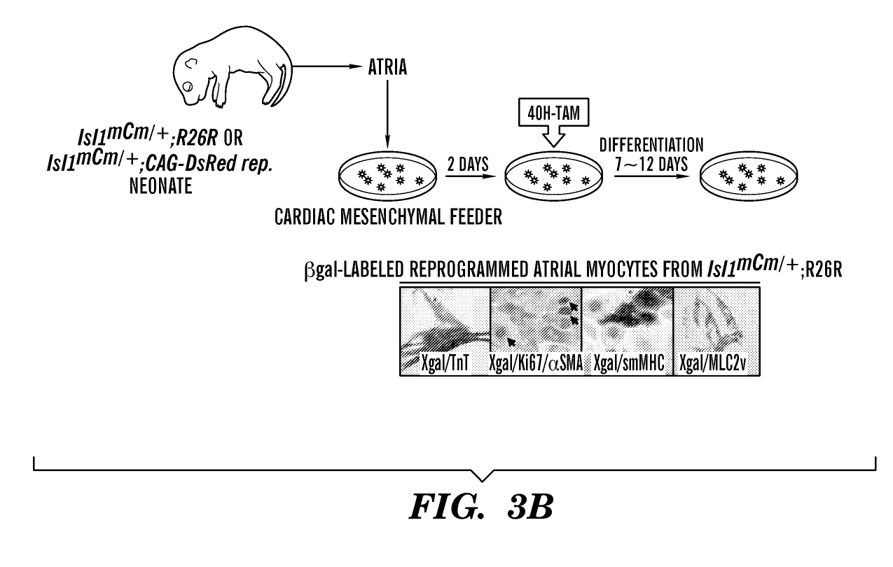

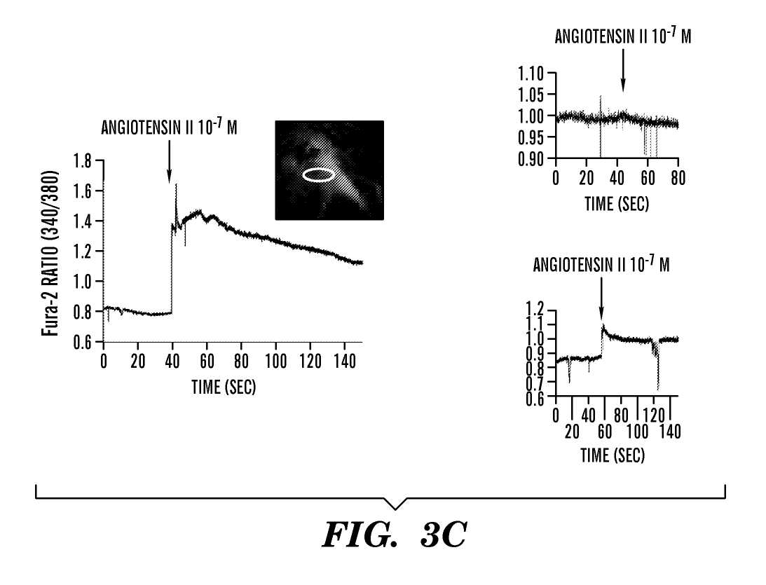

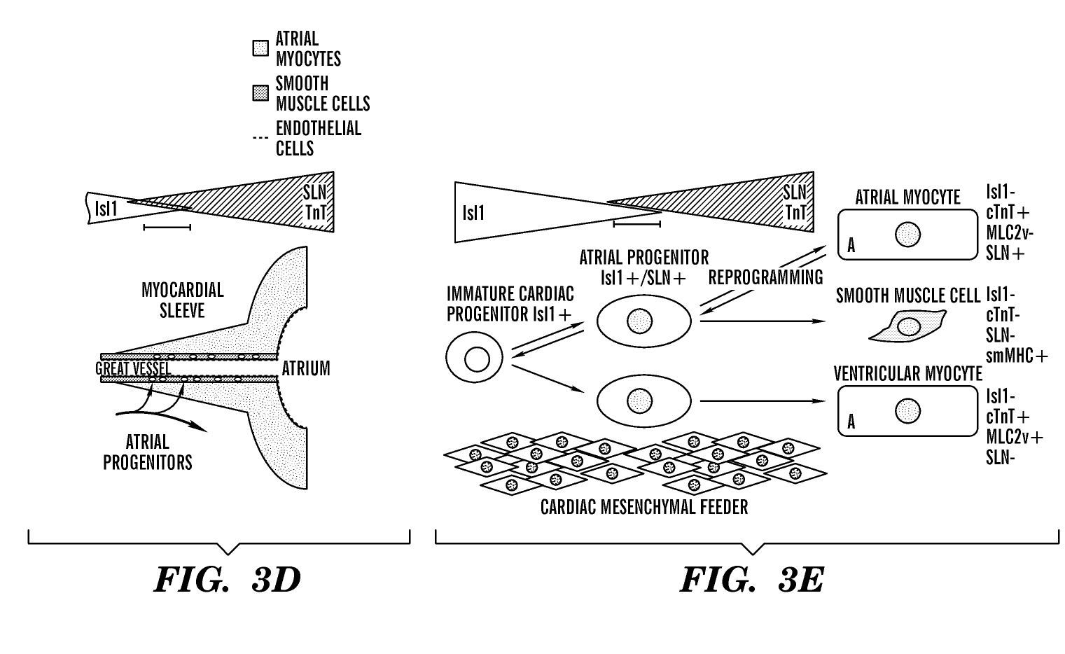

For further confirmation, the inventors dissected the inflow region of the atrium from SLN^{cre/+}; R26R adult mice, isolated the βgal-labeled cells onto fibronectin-coated dishes, and double-stained for Xgal/smMHC. Electron microscopic analysis revealed that approximately 5-10% of the smooth muscle cells with non-striated myofilaments are labeled with Xgal deposits. These data demonstrate that the developmental contribution of posterior secondary heart field/venous pole lineage into smooth muscle cells in the course of migration from splanchnic mesoderm (FIG. 3C). This discovery is a good contrast with anterior heart field/arterial pole subpopulation of Isl1⁺ progenitors that contribute to smooth muscle cells in the base of the ascending aorta¹².

Lineage tracing experiments indicated that SLN-Cre is active at least from E10.5 (data not shown), whereas Isl1 expression continues until E13.5 in the atria¹⁰, demonstrating that there is a spatial and temporal overlap of these two markers in the forming atria. Double staining for Xgal and Isl1 on SLN^{cre/+}×R26R embryos revealed the Isl1/SLN double positive cells at the cellular level in the forming atria (data not shown). At E10.5, immature cardiac progenitors with strong Isl1 expression were found in splanchnic mesoderm (data not shown). In particular, the inventors identified Isl1+/SLN+ atrial progenitors in heart section from SLN^{cre/+}; R26R embryo at E10.5 and E13.5 as cells which were double-stained for Xgal and Isl1 (data not shown). Progenitors in the splanchnic mesoderm were identified to strongly express Isl1 but not SLN. At E10.5, most of Isl1-positive cells in forming atrium were identified to be negative for Xgal, whereas septal atrial myocytes were identified to strongly express Isl1, however a weaker level of Isl1 was detected in Xgal-positive cells in atrial free wall and sinus venosus (data not shown). At E13.5, most of the atrial myocytes were identified to express SLN. Additionally, Isl1-positive immature cardiac progenitors in dorsal mesocardium were identified to migrate into the septum and begin to express SLN while maintaining Isl1 expression (data not shown), although as they migrate toward cushion, atrial cells gradually lose Isl1 expression. Thus, the inventors discovered that a subset of cardiac cells in atrial chamber and sinus venosus already start to express SLN, and some are dual-positive for Isl1 and SLN (data not shown). At E13.5, most of the atrial myocytes were already positive for βgal activity and Isl1+/SLN+ cells were still found in dorsal mesocardium and atrial septum (data not shown). This mediastinal myocardium population^{10, 13-16} gradually loose Isl1 expression as they migrate towards the cushion and progressively acquire SLN expression. These data demonstrate that Isl1+/SLN+ double positive cells (atrial progenitors) in atrial chamber¹⁷, sinus venosus (likely including Tbx18+ population¹⁸) and mediastinal myocardium represent transitional cell populations that are already committed to the atrial lineage but still maintain high proliferative and migratory capacity.

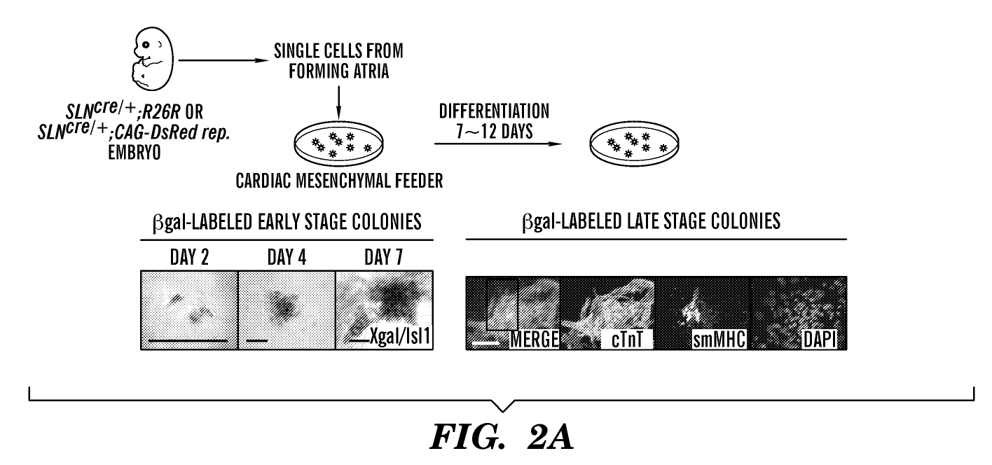

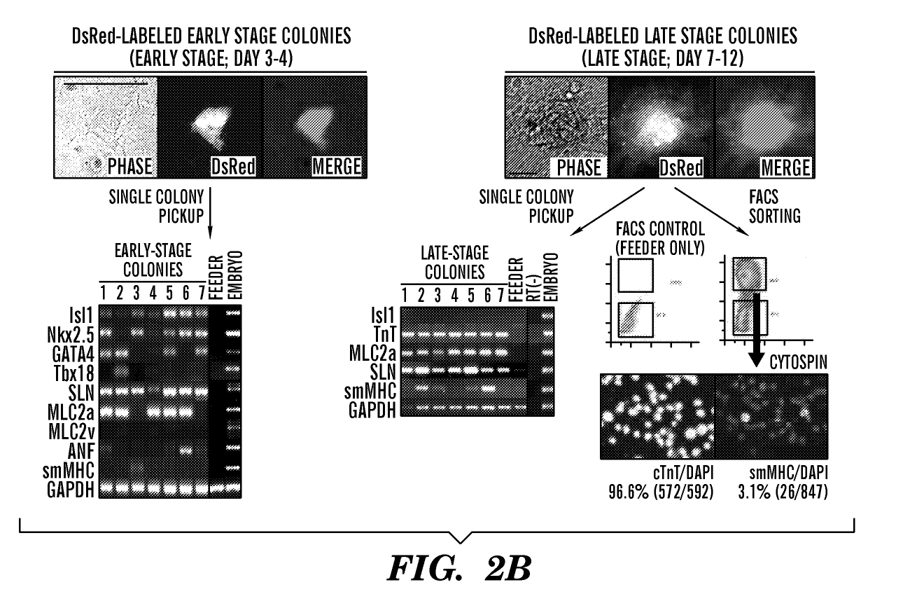

To examine whether cardiac and smooth muscle lineages can originate from a single common Isl1+/SLN+ atrial progenitor, the inventors then dissected forming atrial tissue from E9.5 SLN^{cre/+}; R26R embryos, dissociated them into single cells and cultured them at clonal density onto cardiac mesenchymal feeder layers which are an efficient feeder system for cardiac progenitors^{1, 19}. βgal-labeled atrial progenitors grew as clusters, and 80.6% of the clusters were co-stained with Xgal/Isl1 (FIG. 2A and Table 2). Clonally amplified atrial progenitor colonies after 3 days on feeder (early-stage colonies) showed an expression profile characteristic of the early in vivo atrial lineage (FIG. 2B). After differentiation for 7˜12 days in culture, some βgal-labeled cells in the periphery of the single cell-derived colonies escaped from the myocardial lineage and lost cTnT expression (FIG. 2Ab, black arrows). These peripheral cells were co-stained with Xgal/smMHC. 51.3% of the βgal-labeled colonies from E9.5 embryo contained smMHC-positive cells (data not shown). At the cellular level, smMHC expression was found in 3.1% of the cells differentiated from cultured atrial progenitors (FIG. 2B). Atrial progenitor colonies derived from later stage showed decrease in bipotency (Table 2). These data demonstrate that Isl1+/SLN+ atrial progenitors can be clonally expanded on feeder and differentiated into smooth muscle cells as well as cardiomyocytes in culture.

TABLE 2
Quantification of atrial progenitor colonies from smooth muscle
differentiation. Atrial progenitor colonies derived from E9.5, 12.5 and 15.5 SLN^{cre/+}x R26R
embryos were scored for the number of Isl1 positive blue colonies per total blue colonies and
smMHC-positive βgal-labeled colonies per total blue colonies. Note that Isl1 is expressed in the
atrial cellcolonies derived from E15.5 atria where Isl1 is already downregulated in vivo.

| | | E12.5 | E15.5 |
|---|---|---|---|
| Isl1 expression | 80.6% (29/36) | 55.4% (31/56) | 45.5% (10/22) |
| (positive/total) | | | |
| SMC differentiation | 51.3% (20/39) | 19.6% (14/51) | 17.9% (10/39) |
| (Positive/total) | | | |

To examine the requirement of atrial progenitors in anchoring great veins and atrial chambers, the inventors then ablated atrial progenitor by overexpressing β-catenin in atrial lineage. β-catenin signaling is known to inhibit the differentiation of cardiac progenitors in multiple steps^{6, 20-25}. Constitutive overexpression of β-catenin in SLN^{Cre/+} βcat^{ex3/+} embryo²⁶ resulted in significantly smaller atria and markedly narrower proximal vena cava at E11.5 (data not shown). The inventors next demonstrated that atrial cells can be ablated by β-catenin overexpression. In particular, SLN^{Cre/+}; βcat^{ex3/+} embryos were discovered to have a significantly smaller atria and vena cava as compared with control littermates on whole mount and serial section analysis (data not shown). One out of the three mutants examined also showed hematoma possibly leaking from the boundary of inflow and atria. One of the three mutant embryo examined displayed a large hematoma in the pericardial sac, representing leakage of blood from the boundary of the atrial chamber and the inflow tract. These data demonstrate that the requirement of atrial progenitors in the anchoring of the atrial chamber to the inflow tract.

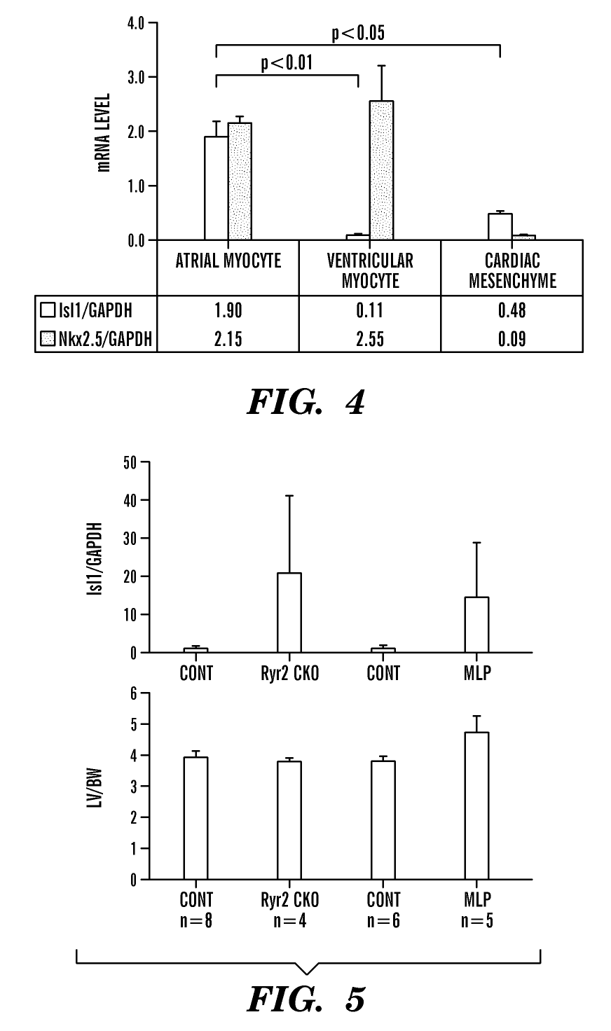

Interestingly, Isl1⁺/Xgal⁺ colonies were also obtained from the atria at E15.5 when Isl1 expression is already downregulated in vivo (Table 2), demonstrating that atrial cells re-expressed upon culture. Following this discovery, the inventors investigated the plasticity of postnatal atrial myocytes. The inventors first examined the Isl1 re-expression of neonatal atrial myocytes using atria-specific Cre strain. Although the postnatal atrial cardiomyocytes do not express Isl1 in vivo, 40.4% of primary neonatal atrial myocytes labeled with βgal or DsRed re-expressed Isl1 on feeder (Table 3). If one considers that contamination of non-myocyte is 10% in the atrial myocyte fraction, in vitro qPCR analysis (FIG. 4) indicates that majority (97.6%) of the Isl1 mRNA is derived from atrial myocytes and only 2.4% from non-myocytes. Thus, this population is different from residential Isl1+ progenitors embedded in cardiac mesenchyme⁵. The Isl1 re-expression is due to epigenetic activation of Isl1 promoter, because ChIP assay on Isl1 promoter indicates that trimethylation level of Lysine 27 of Histone H3 becomes significantly lower after Isl1 re-activation (FIG. 3A). Isl1 re-activation also takes place in injured atria. Isl1 staining on rat atrial cryoinjury model resulted in a few Isl1 positive cells within cardiomyocytes in peri-injury zone (data not shown). In particular, using double immunohistochemistry with Isl1 and pH3 on rat atrial cryoinjury model where rat atrial appendages were ligated and examined 3 days following surgery, the inventors detected Isl1-positive cells and pH3-positive cells in the atrial myocytes in the peri-injury zone 3 days after surgery. This Isl1 re-induction was also confirmed by qPCR analysis of the atrial samples from two genetic ablation models (FIG. 5). On the other hand, Isl1 was not re-expressed in primary culture of MLC2v^{cre/+}; R26R ventricular mycoytes (FIG. 4) or in rat in vivo cryoinjury (data not shown).

Re-expression of Isl1 raises the possibility that the postnatal atrial myocyte can be reprogrammed to a more immature and proliferative stage²⁷ and partially reacquire property of atrial progenitor. Utilizing proliferation markers in labeled atrial myocytes, the inventors demonstrated that Xgal/pH3 and Xgal/Ki67 double positive cells indicated that the atrial myocytes were re-entering the cell cycle (data not shown). Furthermore, triple staining of DsRed-labeled neonatal atrial myocytes for DsRed (atrial lineage), BrdU (proliferation) and Isl1 revealed that half of the Isl1/DsRed double positive cells were BrdU-positive and that none of the Isl1-negative atrial myocytes incorporates BrdU (data not shown). For example, the inventors examined re-expression of Isl1 in neonatal atrial myocytes. In particular, βgal-labeled neonatal atrial myocytes cultured on cardiac mesenchymal feeder were discovered to co-express both Xgal and Isl1 after 3 days in culture (data not shown), with an increase in the number of double positive cells as the clusters of neonatal atrial myocytes grew. The inventors next demonstrated cell cycle reentrance of Isl1-reexpressing atrial myocytes by treating DsRed-labeled neonatal atrial myocytes with BrdU and triple-stained with mouse anti-Isl1 (green), rabbit anti-DsRed (red) and rat anti-BrdU (blue) antibodies. The inventors discovered that clusters contained Isl1-re-expressing atrial myocytes with BrdU incorporation (Isl1+/DsRed+/BrdU+) and Isl1+ +/BrdU-cells, which are Isl1-positive atrial cells without mitotic activity. Isl1+/DsRed−/BrdU+ cells were identified to be non-labeled atrial cells or non-cardiac Isl1-positive cells. Strong correlation between Isl1 re-expression and BrdU incorporation demonstrated that reversion to the Isl1-positive stage is strongly associated with cell cycle re-entrance in atrial lineages.

TABLE 3
Percentage of Isl1 (+) and BrdU (+) cells within DsRed population.
Demonstrates the correlation of Isl1-positivity and BrdU incorporation within the atrial cell
population. As discussed above, 40.4% of the labeled atrial myocytes reexpress Isl1. About half
Isl1-positive atrial cells incorporates BrdU. Differentiated atrial myocytes (Isl1-negative, DsRed-
positive atrial myocytes) never incorporated BrdU in 4 independent experiments. P<000.5

| | Isl1(+) | Isl1(−) |
|---|---|---|
| BrdU(+) | 19.9% | 0.0% |
| BrdU(−) | 20.5% | 59.7% |
| Total | 40.4% | 59.7% |

To examine whether the Isl1+ post-natal atrial cells can redifferentiate into multiple lineages, the inventors employed temporal labeling of Isl1 using Isl1-mer-Cre-mer knock-in mice (FIG. 3B). Neonatal atrial myocytes isolated from Isl1mCm/×R26R breeding were cultured on mesenchymal feeder and stimulated with 4OH-TAM for 48 hours, so that only the atrial cells re-expressing Isl1 are labeled with βgal. Again, 97.4% of the Isl1 level in this culture is derived from atrial lineage by calculation (FIG. 4). βgal-labeled atrial cells were able to redifferentiate into smMHC-positive smooth muscle cells and MLC2v-positive ventricular myocytes after 7-14 days (FIG. 3B). The phenotypical conversion was also evident using βgal- and DsRed-labeled atrial myocytes derived from SLN^{cre/+} R26R and SLN^{cre/+}; CAG-DsRed reporter neonates (data not shown). The phenotypical conversion of atrial myocytes into smooth muscle cells and ventricular myocytes was determined by isolating primary atrial myocytes from SLN^{cre/+}×R26R SLN^{cre/+}×CAG-DsRed reporter neonates, and analysis by stained with Xgal followed by immunostaining for cTnT, αSMA or smMHC (data not shown). The expression of smMHC was detected in 1.2% of the labeled cells after 7-14 days' culture. To examine the functional property of these transdifferentiated cells, [Ca²⁺]i transient in response to Angiotensin-II was measured in DsRed-labeled atrial cells. In 3 out of 30 DsRed-labeled cells examined that are not apparently beating, the inventors discovered that Angiotensin-II elicited a pattern of Fura-2 ratio change that was similar to that observed in cultured aortic smooth muscle cells (FIG. 3C). Engraftment of reprogrammed atrial myocytes into ventricular wall are useful for cell replacement therapy (data not shown). Therefore, Isl1 re-activation accompanies epigenetic change, cell cycle reentrance and acquisition of reprogramming capability into functional smooth muscle and ventricular myocytes. The inventors herein have discovered that differentiated atrial myocytes can be reprogrammed into their multipotent Isl1-progenitor state in culture (FIG. 3E).

The inventors also used Xgal staining of a SLNcre/+; R26R neonatal neonatal heart for atrial lineage tracing to identify if Xgal is restricted in atrial lineage throughout embryonic and postnatal stages. The inventors discovered that the endocardial and epicardial layers were negative for Xgal staining (data not shown), even when the looked at whole mount Xgal staining of SLNcre/+; R26R embryonic and adult heart showing the extension of myocardial sleeves.

The inventors discovered cell cycle reentrance of primary neonatal atrial myocytes isolated from SLN^{cre/+}×R26R, were double positive for Xgal/pH3 or Xgal/Ki67, demonstrating the occurrence of cycle reentrance of postnatal atrial myocytes (data not shown). Additionally, the inventors demonstrated that reprogrammed atrial progenitor-like cells that were engrafted into ventricular wall were viable. For instance, βgal-labeled neonatal atrial myocytes were cultured on feeder and injected into ventricular wall of SCID mice and were discovered to have a ventricular myocyte phenotype after 28 days.

Taken together, the inventors have discovered a unique subset of Isl1⁺ progenitors that give rise to Isl1⁺/SLN⁺ atrial lineages, including the components of the SA nodal conduction system, venous valvular structures, vascular smooth muscle in the inflow tract, and the atrial chambers themselves. From a developmental perspective, the Isl1⁺/SLN⁺ progenitors represent components of the posterior region of the secondary heart field²⁸, and retain proliferative activity²⁹ and bipotency late during cardiogenesis, which relates to their generation of the myocardial/smooth muscle sleeves that serve as the junctional boundary to fuse the great vessels and the cardiac chambers into a functional syncytium. Interestingly, there are several diseases of the atrium and inflow tract, including a common form of congenital heart disease where the pulmonary venous inflow tract is ectopic or absent, and atrial fibrillation that relates to a reemergence of ectopic electrical activity in the pulmonary veins. The discovery of bipotent Isl1⁺/SLN⁺ atrial progenitors, and demonstration of the reversibility of bipotency and the subsequent trans-differentiation of differentiated atrial cells to alternative muscle phenotypes, demonstrates that a subset of atrial diseases are likely to be a result of dys-regulation of bipotency step in atrial lineages.

In addition, the ability to reprogram and clonally expand post natal atrial cells into Isl1+ progenitors, in particular into Isl1⁺/SLN⁺ atrial progenitors on cardiac mesenchymal feeder layers, and to trigger their subsequent differentiation into distinct muscle subtypes, demonstrates an important role for these cells in specific applications for regenerative therapy in the setting of congenital heart diseases^{30, 31}.

### REFERENCES

The references cited herein and throughout the application are incorporated herein in their entirety by reference.
1. Moretti, A. et al. Multipotent embryonic isl1+ progenitor cells lead to cardiac, smooth muscle, and endothelial cell diversification. Cell 127, 1151-65 (2006).
2. Wu, S. M. et al. Developmental origin of a bipotential myocardial and smooth muscle cell precursor in the mammalian heart. Cell 127, 1137-50 (2006).
3. Kattman, S. J., Huber, T. L. & Keller, G. M. Multipotent flk-1+ cardiovascular progenitor cells give rise to the cardiomyocyte, endothelial, and vascular smooth muscle lineages. Dev Cell 11, 723-32 (2006).
4. Garry, D. J. & Olson, E. N. A common progenitor at the heart of development. Cell 127, 1101-4 (2006).
5. Laugwitz, K. L. et al. Postnatal isl1+ cardioblasts enter fully differentiated cardiomyocyte lineages. Nature 433, 647-53 (2005).
6. Qyang, Y. et al. The Renewal and Differentiation of Isl1+ Cardiovascular Progenitors Are Controlled by a Wnt/β-Catenin Pathway. Cell Stem Cell 1, 165-179 (2007).
7. van Laake, L. W., Hassink, R., Doevendans, P. A. & Mummery, C. Heart repair and stem cells. J Physiol 577, 467-78 (2006).
8. Odermatt, A. et al. Characterization of the gene encoding human sarcolipin (SLN), a proteolipid associated with SERCA1: absence of structural mutations in five patients with Brody disease. Genomics 45, 541-53 (1997).
9. Minamisawa, S. et al. Atrial chamber-specific expression of sarcolipin is regulated during development and hypertrophic remodeling. J Biol Chem278, 9570-5 (2003).
10. Sun, Y. et al. Islet 1 is expressed in distinct cardiovascular lineages, including pacemaker and coronary vascular cells. Dev Biol 304, 286-96 (2007).
11. Pashmforoush, M. et al. Nkx2-5 pathways and congenital heart disease; loss of ventricular myocyte lineage specification leads to progressive cardiomyopathy and complete heart block. Cell 117, 373-86 (2004).
12. Waldo, K. L. et al. Secondary heart field contributes myocardium and smooth muscle to the arterial pole of the developing heart. Dev Biol 281, 78-90 (2005).
13. Soufan, A. T. et al. Reconstruction of the patterns of gene expression in the developing mouse heart reveals an architectural arrangement that facilitates the understanding of atrial malformations and arrhythmias. Circ Res 95, 1207-15 (2004).
14. Anderson, R. H., Brown, N. A. & Moorman, A. F. Development and structures of the venous pole of the heart. Dev Dyn 235, 2-9 (2006).
15. Mommersteeg, M. T. et al. Two distinct pools of mesenchyme contribute to the development of the atrial septum. Circ Res 99, 351-3 (2006).
16. Moorman, A. F., Christoffels, V. M., Anderson, R. H. & van den Hoff, M. J. The heart-forming fields: one or multiple? Philos Trans R Soc Lond B Biol Sci 362, 1257-65 (2007).
17. Galli, D. et al. Atrial myocardium derives from the posterior region of the second heart field, which acquires left-right identity as Pitx2c is expressed. Development (2008).
18. Christoffels, V. M. et al. Formation of the venous pole of the heart from an Nkx2-5-negative precursor population requires Tbx18. Circ Res 98, 1555-63 (2006).
19. Kruithof, B. P., van den Hoff, M. J., Wessels, A. & Moorman, A. F. Cardiac muscle cell formation after development of the linear heart tube. Dev Dyn 227, 1-13 (2003).
20. Naito, A. T. et al. Developmental stage-specific biphasic roles of Wnt/beta-catenin signaling in cardiomyogenesis and hematopoiesis. Proc Natl Acad Sci USA 103, 19812-7 (2006).
21. Cohen, E. D. et al. Wnt/beta-catenin signaling promotes expansion of Isl-1-positive cardiac progenitor cells through regulation of FGF signaling. J Clin Invest 117, 1794-804 (2007).
22. Klaus, A., Saga, Y., Taketo, M. M., Tzahor, E. & Birchmeier, W. Distinct roles of Wnt/beta-catenin and Bmp signaling during early cardiogenesis. Proc Natl Acad Sci USA 104, 18531-6 (2007).
23. Kwon, C. et al. Canonical Wnt signaling is a positive regulator of mammalian cardiac progenitors. Proc Natl Acad Sci USA 104, 10894-9 (2007).
24. Tzahor, E. Wnt/beta-catenin signaling and cardiogenesis: timing does matter. Dev Cell 13, 10-3 (2007).
25. Ueno, S. et al. Biphasic role for Wnt/beta-catenin signaling in cardiac specification in zebrafish and embryonic stem cells. Proc Natl Acad Sci USA 104, 9685-90 (2007).
26. Harada, N. et al. Intestinal polyposis in mice with a dominant stable mutation of the beta-catenin gene. Embo J 18, 5931-42 (1999).
27. Poss, K. D., Wilson, L. G. & Keating, M. T. Heart regeneration in zebrafish. Science 298, 2188-90 (2002).
28. Buckingham, M., Meilhac, S. & Zaffran, S. Building the mammalian heart from two sources of myocardial cells. Nat Rev Genet 6, 826-35 (2005).
29. Soufan, A. T. et al. Regionalized sequence of myocardial cell growth and proliferation characterizes early chamber formation. Circ Res 99, 545-52 (2006).
30. Parmacek, M. S. & Epstein, J. A. Pursuing cardiac progenitors: regeneration redux. Cell 120, 295-8 (2005).
31. Smart, N. et al. Thymosin beta4 induces adult epicardial progenitor mobilization and neovascularization. Nature 445, 177-82 (2007).
32. Knoll, R. et al. The cardiac mechanical stretch sensor machinery involves a Z disc complex that is defective in a subset of human dilated cardiomyopathy. Cell 111, 943-55 (2002).
33. Sohal, D. S. et al. Temporally regulated and tissue-specific gene manipulations in the adult and embryonic heart using a tamoxifen-inducible Cre protein. Circ Res 89, 20-5 (2001).
34. Xu, H., Chen, L. & Baldini, A. In vivo genetic ablation of the periotic mesoderm affects cell proliferation survival and differentiation in the cochlea. Dev Biol 310, 329-40 (2007).
35. Ichinose, F. et al. Cardiomyocyte-specific overexpression of nitric oxide synthase 3 prevents myocardial dysfunction in murine models of septic shock. Circ Res 100, 130-9 (2007).

1. A method for isolating atrial progenitors, the method comprising contacting a population of progenitor cells with at least one agent reactive to Islet 1 and SLN, and separating reactive positive cells from non-reactive cells.

2. The method of claim 1, further comprising introducing a reporter gene operatively linked to the regulatory sequence for Islet1 and SLN and separating the reactive positive cells expressing the reporter gene from non-reactive cells.

3. The method of claim 1, wherein the atrial progenitors are capable of differentiating into cells with a muscle cell or cardiomyocyte phenotypes.

4. The method of claim 3, wherein the cardiomyocyte phenotypes is an atrial myocyte.

5. The method of claim 4, wherein the atrial myocyte is a cTnT-positive, SLN-positive, Islet1-negative and MLC2v-negative atrial myocyte.

6. The method of claim 3, wherein the muscle cell phenotype is a smooth muscle cell.

7. The method of claim 6, wherein the smooth muscle cell is a smMHC-positive, Islet1-negative, cTnT-negative and SLN-negative smooth muscle cell.

8. The method of claim 1, wherein the agent is a nucleic acid agent or protein agent which is reactive to a nucleic acid encoding Islet 1 or SLN.

9. The method of claim 1, wherein the agent is a nucleic acid agent or protein agent which is reactive to an expression product of the nucleic acid encoding Islet1 or SLN.

10. The method of claim 2, wherein the reporter gene encodes fluorescence activity and/or chromogenic activity.

11. A method to generate a Isl1+/SLN+ atrial progenitor cell, the method comprising culturing at least one atrial myocyte cell or at least one Isl1+ progenitor cell in the presence of a cardiac messenchymal cell feeder layer for a sufficient period of time for the at least one atrial myocyte cell or the at least one Isl1+ progenitor cell to differentiate into Isl1+/SLN+ atrial progenitor cell.

12. The method of claim 11, wherein the atrial myocyte is a mature atrial myocyte cell.

13. The method of claim 12, wherein the mature atrial myocyte cell is a cTnT-positive (cTNT⁺), SLN-positive (SLN⁺), Islet1-negative (Isl1⁻) and MLC2v-negative (MLC2v⁻) mature atrial myocyte.

14. The method of claim 11, wherein the at least one atrial myocyte cell or at least one Isl1+ progenitor cell is from a mammal.

15. The method of claim 14, wherein the mammal is a human.

16. The method of claim 11, wherein the at least one atrial myocyte cell is a genetically modified atrial myocyte cell.

17. The method of claim 11, wherein the at least one Isl1⁺ progenitor cell is a genetically modified Isl1⁺ progenitor cell.

18. A composition comprising an isolated population of Islet1⁺, SLN⁺ atrial progenitor cells.

19. The composition of claim 18, wherein the Islet1⁺, SLN⁺ atrial progenitor cells are generated according to the methods of claims 11-17.

20. The composition of claim 18, wherein the composition is subsequently cryopreserved.


SEQUENCE LISTING (7 sequences)

SEQ ID NO: 1 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 2 - DNA, 2448 bp, Homo sapiens
tgaaggaagaggaagaggaggagagggaggccagagccagaacagcccggcagcccgggcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggaatcctagatccgcgagggcgcggcgcagccgagcagcggctctttcagcattggcaaccccaggggccaatatttcccacttagccacagctccagcatcctctctgtgggctgttcaccagctgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccggccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactttaaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaaaaa

SEQ ID NO: 3 - DNA, 2447 bp, Homo sapiens
gaaggaagaggaagaggaggagagggaggccagagccagaacagcccggcagcccgggcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggaatcctagatccgcgagggcgcggcgcagccgagcagcggctctttcagcattggcaaccccaggggccaatatttcccacttagccacagctccagcatcctctctgtgggctgttcaccagctgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccggccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactttaaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaaaaa

SEQ ID NO: 4 - protein, 31 aa, Homo sapiens
MGINTRELFLNFTIVLITVILMWLLVRSYQY

SEQ ID NO: 5 - DNA, 716 bp, Homo sapiens
agacagcctgggagggagaaggagttggagctcaagttggagacagcgaggagaaacctgccatagccagggtgtgtctttgatcctcttcaggaggtgaggacaagccagaggtccttggtgtgccctcagaaatctgcctgcagttctcaccaagccgctgtgaaaatggggataaacacccgggagctgtttctcaacttcactattgtcttgattacggttattcttatgtggctccttgtgaggtcctatcagtactgagaggccatgccatggtcctgggattgactgagatgctccggagctgcctgctctatgccctgagaccccactgctgtcattgtcacaggatgccattctccatccgagggcacctgtgacctgcactcacaatatctgctatgctgtagtgctaggattgattatgtgttctccaaagatgctgctcccaagggctgccaagtgtttgccagggaacggtagatttattccccaactcttaactgaaaatgtgttagacaagccacaaagttaaaattaaactggattcatgatgatgtaggattgttacaagcccctgatctgtctcaccacacatcccttcaacccacacggtctgcaaccaaactctaattcaacctgccagaaggaatgttagaggaagtctttgtcagcccttatagctatcatgtgaataaagttaagtcaacttc

SEQ ID NO: 6 - DNA, 738 bp, Homo sapiens
agtccagacagcctgggaggggagaaggagttggagctcaagttggagacagcgaggagaaacctgccatagccagggtgtgtctttgatcctcttcaggaggtgaggagaagccagaggtccttggtgtgccctcagaaatctgcctgcagttctcaccaagccgctgtgaaaatggggataaacacccgggagctgtttctcaacttcactattgtcttgattacggttattcttatgtggctccttgtgaggtcctatcagtactgagaggccatgccatggtcctgggattgactgagatgctccggagctgcctgctctatgccctgagaccccactgctgtcattgtcacaggatgccattctccatccgagggcacctgtgacctgcactcacaatatctgctatgctgtagtgctaggattgattatgtgttctccaaagatgctgctcccaagggctgccaagtgtttgccagggaacggtagatttattccccaactcttaactgaaaatgtgttagacaagccacaaagttaaaattaaactggattcatgatgatgtaggattgttacaagcccctgatctgtctcaccacacatcccttcaacccacacggtctgcaaccaaactctaattcaacctgccagaaggaatgttagaggaagtctttgtcagcccttatagctatcatgtgaataaagttaagtcaacttcaaaaacaaaaaaaaaa

SEQ ID NO: 7 - DNA, 4687 bp, Homo sapiens
agtccagacagcctgggaggggagaaggagttggagctcaagttggagacagcgaggagaaacctgccatagccagggtgtgtctttgatcctcttcaggtaactgcaggatttttatgcttagcatacggtgtgtgtgtgtgtacaagctgcaaatgagaaaggcagaggaatgtttgcatttcttaatgaaataggcaatgccccttggatacagcatgccttaagatataaagtgagtactattgctttctgttttcctttttttttctcctaagggtatgcattttctcccttcctagtaaaaaaaaattttttttctttcttgtattatggaagatggtcttttttctatcttctctaagtctccattcaaagaaagccaatgtcaaacttcctgcacaatttattcagagctttttgggttttagttcatccatttgcaatcactggatcatatggcatgaccttgacttcagggaatgtttaaaatgaaacccaaagtgtctttattggttaaaagcagaaaaatggaacttggctgatctgaatttctgcctaactggttcattcttcctaaagtgtataaatgtgtgtgaaaggaatgttcacaccacaccttacagttagcatatgtgttgattgaattgaattcactagaaattctcaccttaactcttcatgttttattcatctaaaatcacataaaacaacaaatattcaaggtcaccattaaagtgcaagaagtagtaagaggtaaatcccaagaaagaaagaagtgtgatgggtagcctgctaattcagaaatgactgaaaaaaaaaaagttaagagtgcataaacatacacagtttaaacaataactgaaaggcaacacttctataattatcatccaggtcaagaaacagatcattgccagcacccagaagcccattcttgatcaacccccattctgccccaagatgttgggatcctcttgtccctgttattctttataatttgaccatctatgtctgcatgcctaaacaatatgattaacctccctatttttgacatcatataaatggagtcatattctctgtttcttttgtaaattcttctttcacccaacaattatatttgtgagatctacccattttctgtgtagctatagttcatatattttctttgctttatgaaattcaaatgtataactataccacaatttatcatccattctgctgttgataaacatttgagttgtttccagtttggagttattgtgataatattgctgtgaactctttatttatgtaacctggcacgcacgtccttgactgtcttaaggtagaagtgaaatgccagatattagagaatgaacattttcaacattaattctaagaaagctaaattgttttccaaagtggttgaaaatttatgctaactttgtttagccaggccagttaattgcagtgacaattcacttgtgtcataactcctaggaattctagtaactagttccagtctgctaattctagctttgcctctaacttgttatctgtcattacacaaacatcttaatctcctctctctaaaagtcagtttctcagctgtaatcttgagaaagataaaaactgcagtaacacttgcctgagttgttgcgaaggtaacacaaacagagctgtggaacagctttcaatagtatttttctcattttctgagtatatcctgttggatcactatccatgattcccatgacttaacagcttaatttaataggcagatttggtgggaccgttgttttgctctccttgaattgttttgaccattataagccaggcttcatattttctgcttatctctgcatttcttgagaacttacccaaatagatggacatttggactatggttttatttcattgataagtttataagaaagagtctgtcttcgtttctcattcctttgcctccaggcaccttggcaagactcattaccgttctgttctcctctgctcttttgtacattctcataaatcctttttccttgatccattcattttattctgtttactttctcctccatgcaaatctttgtaaagattttctattttttcccctttttattttcttggagagacagggtcttgctgtgttatccaggctggtcttgaactcctaggctcaagtgatcctcccactccagcctcccaaagggctgggattagaggcatgagccaccacgcccagccatttgtaatggttttcattggcaatttactatttccttcatgctgaggtctaaaagatggataataattaaaagggagatgaaatataaatctgtcatcatttggctatatatctactggttggcaccatctgatttgaacttgtatttttttttgagacagggtctcattatgttgcctaagctggtcttaaactcctaggctcaagggatcctcccacctcagcttcccaattagctaggactacaggtaccgactgccatgcctggctgaacctgtatgttatgccaattacacaatatgaaaaatgatcattcagttggcatttatgcaattaacaatttggtccattgataatataatcaatacagtaattacaattaataattcagtcatgctagatagtttaaaaataccactcagaccaaaagcttgctaacaaccctttcagcctcttgaactctcacctttagcaaacacacaaaataccctttctccctaaactttcactctacagtagactttggtcctaaatgtctaaaacatagatatcgaattatctaaaatatataaatctttaatttcatttaggctgcataaataatgtgcaatttacaccatctcactgaagacaatagtttgatgacttcattgcacatgttctcaaagtatgatttcctgtatggacaatctgtacatactatgaagataccatccctgctctttgaaatgtttacaatctaggcacatcctggacctgactaaaaatagctggcacaagcccttgttgaaaccgaagtacccatgaagttaacacttgctctttcggctgaatgcctgttatgccaggggcttttggttgatcccagtaggctatgtgcccaatgcaggacttgtcagaggccatgcagctgttgaaagtactttctgctttccgtagagttagatgaagacctacagcagcctgaggcctgaaagattatggcatcccacttatctatgtaattcaaagcaagaacaatactaaaccataaaatttttttgttttggtacccctgcttattgatactcttaagaaaagtatacacatgcatacgcacacacgcaaacacacacagaagttggagaaagaaaaatgtaatatttcacattttatgaaaagcaccaatacgatcatcacaggaaaaaagtcaggacaaaaatggtggtctttatgacatcaggcagagaaatcaggctctctctctgtaatcattagggctcttcagagaaacataactaatagaatgtgttgtgttgaggggattggtgtctgtttttaaggaattggcttgtgcgattgcagagactggcaagtccaaaatctgcagggtgggctggtaggctggagacttagggaagtctccagtccaaagactgtcaggaggctacatgtcccagatcaaggtgttggcacagttggtttcttctgagtcctttctttggcttgtaaatgcccatcttctccttgtgttttcacatggtcttcccctctgcatgtgtctgtgtcctaatctcttctgataaagacactagtcatattggattagggccctccttaattacctaattttaacttatttacctcttaaaagaccctatcttcaaacacagtcacattctgagatactgtgagctagggctttgacatgtagatttttgggggacacaatttagcccatcacactctccttttccacaacacttctgtttcctttgaggaaagaacgagcattgttatacaggaatgcccattaatctccttgtgttttctttgttatgttttatcaggaggtgaggagaagccagaggtccttggtgtgccctcagaaatctgcctgcagttctcaccaagccgctgtgaaaatggggataaacacccgggagctgtttctcaacttcactattgtcttgattacggttattcttatgtggctccttgtgaggtcctatcagtactgagaggccatgccatggtcctgggattgactgagatgctccggagctgcctgctctatgccctgagaccccactgctgtcattgtcacaggatgccattctccatccgagggcacctgtgacctgcactcacaatatctgctatgctgtagtgctaggattgattatgtgttctccaaagatgctgctcccaagggctgccaagtgtttgccagggaacggtagatttattccccaactcttaactgaaaatgtgttagacaagccacaaagttaaaattaaactggattcatgatgatgtaggattgttacaagcccctgatctgtctcaccacacatcccttcaacccacacggtctgcaaccaaactctaattcaacctgccagaaggaatgttagaggaagtctttgtcagcccttatagctatcatgtgaataaagttaagtcaacttcaaaaacaa